(19) United States
(12) Patent Application Publication
(10) Pub. No.: US 2025/0320545 A1
(43) Pub. Date: Oct. 16, 2025

(54) IMPROVED CRISPR-CAS TECHNOLOGIES

(71) Applicant: Sherlock Biosciences, Inc., Watertown, MA (US)

(72) Inventors: Pradeep Ramesh, Washington, DC (US); William Jeremy Blake, Winchester, MA (US); Brendan John Manning, Brighton, MA (US)

(21) Appl. No.: 18/292,859

(22) PCT Filed: Jul. 26, 2022
(86) PCT No.: PCT/US22/38352

§ 371 Date: Jan. 26, 2024

Related U.S. Application Data
(60) Provisional application No. 63/225,802, filed on Jul. 26, 2021.

(51) Int. Cl. C12Q 1/6823; C12N 9/22; C12N 15/11; C12Q 1/682; C12Q 1/70

(52) U.S. Cl. CPC C12Q 1/6823; C12N 9/226; C12N 15/11; C12Q 1/682; C12Q 1/701; C12N 2310/20

(57) ABSTRACT

The present disclosure provides improved CRISPR-Cas proteins (e.g., improved thermostability).

### CROSS REFERENCE TO RELATED APPLICATIONS

This application claims priority to U.S. Provisional Patent Application No. 63/225,802 filed Jul. 26, 2021, the entire content of which is hereby incorporated by reference.

### SEQUENCE LISTING

In accordance with 37 CFR 1.52 (e) (5), the present specification makes reference to a Sequence Listing submitted electronically in XML format and is hereby incorporated by reference in its entirety. Said XML copy, created on Sep. 9, 2022, is named 2013065-0592_SL.xml and is 31.5 kilobytes in size.

### BACKGROUND

A variety of clustered regularly interspaced short palindromic repeats-CRISPR-associated (“Cas”) proteins have been discovered to have collateral (trans) cleavage activity and improved thermostability useful in, for example, detection (e.g., diagnostic) systems to detect particular nucleic acids of interest and as therapeutics (e.g., gene editing). See, for example, review by Sashital Genome Med 2018:10, 32.

### SUMMARY

The present disclosure provides improved CRISPR-Cas proteins characterized by thermostable activity and/or Cas-protein collateral activity. The present disclosure also provides improved guide-RNA technologies.

Among other things, the present disclosure identifies the source of a problem with use of certain Cas enzymes, including specifically in certain collateral activity assays. For example, the present disclosure documents that certain uses, including for example certain such collateral activity assays include a step that involves incubation at an elevated temperature for a period of time, and various Cas enzymes may be insufficiently stable to maintain a sufficient level of activity (e.g., collateral activity) under such conditions. In many embodiments, such a step may be or comprise a nucleic acid extension and/or amplification step.

Alternatively or additionally, the present disclosure provides the insight that particularly desirable embodiments of various reactions that utilize Cas enzymes, including for example certain collateral activity assays, are those that can be performed in a single reaction vessel (i.e., so-called “one pot”) assays. The present disclosure appreciates that Cas enzymes whose activity (e.g., collateral cleavage activity) is insufficiently stable to maintain sufficient activity through any and all elevated-temperature step(s) (which may be or include, for example, one or more nucleic acid extension and/or amplification step(s)) may not be useful in such one-pot assays. The present disclosure furthermore documents that certain Cas protein(s) (e.g., Cas13 and Cas12) are insufficiently stable at relevant temperature(s), e.g., at temperatures at which nucleic acid extension and/or amplification reactions are typically performed (e.g., above about 60-65° C.).

The present disclosure encompasses the recognition that thermostable variants of various Cas proteins (e.g., Cas9) have already been described and/or otherwise made publicly available (see, for example, Mougiakos et al. Nat Commun. 8:1647, 2017). Those skilled in the art are able to compare such thermostable variants with related non-thermostable homologs (e.g., orthologs), in order to assess sequence changes and/or elements that may be necessary and/or sufficient to achieve thermostability, and furthermore can identify such sequence changes and/or elements in other homologs (e.g., orthologs) and/or can introduce them thereinto. Still further, those skilled in the art are well aware of potential sources of naturally-occurring Thermostable Cas Proteins (e.g., in microbes that survive in elevated temperature conditions, such as in sea vents, or are otherwise thermophilic). Thus, those skilled in the art, reading the present disclosure, could readily identify and/or develop appropriate Thermostable Cas Proteins for use as described herein.

In some embodiments, a useful Thermostable Cas Protein is a Cas12 or Cas13 homolog (e.g., ortholog). In some embodiments, a useful Thermostable Cas Protein is a Cas enzyme comprising an amino acid sequence having 80%, 85%, 90%, 99% or 100% sequence identity to any one of SEQ ID Nos. 1-10.

Alternatively or additionally, in some embodiments, a useful Thermostable Cas Protein performs (e.g., its collateral cleavage activity functions sufficiently) at temperatures above about 50° C.; in some embodiments, above a temperature selected from the group consisting of about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In many embodiments, useful Thermostable Cas Protein performs (e.g., its collateral cleavage activity functions sufficiently) at temperatures above about 60° C.

In some embodiments, a useful Thermostable Cas Protein performs (e.g., its collateral cleavage activity functions sufficiently) within a temperature range at which nucleic acid extension and/or amplification reaction(s) are performed; those skilled in the art are well familiar with various such reactions and the temperature ranges at which they are performed, In some embodiments, such a temperature range may be above a temperature selected from the group consisting of about 60° C., about 61° C., about 62° C., about 63° C., about 64° C., 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In some embodiments, a temperature range may be about 60° C. to about 90° C. In some embodiments, a temperature range may be about 60° C. to about 80° C. In some embodiments, a temperature range may be about 60° C. to about 75° C. In some embodiments, a temperature range may be about 65° C. to about 90° C. In some embodiments, a temperature range may be about 60° C. to about 80° C. In some embodiments, a temperature range may be about 60° C. to about 75° C.

Thus, as is set forth herein, in some embodiments, a useful Thermostable Cas Protein is a Cas12 or Cas13 homolog (e.g., ortholog), e.g., a Cas enzyme comprising an amino acid sequence having 80%, 85%, 90%, 99% or 100% sequence identity to any one of SEQ ID Nos. 1-10 that is thermostable at temperatures above about 50° C., and in some embodiments above about 60° C., for example within and/or above about 60-65° C. Those skilled in the art, reading the present disclosure will particularly appreciate that, in some embodiments, a useful Thermostable Cas Protein is a Cas12 (e.g., SEQ ID NOs 1-10, or a variant thereof, for example having at least 90%, 95%, 99% or greater amino acid sequence identity thereto) or Cas13 (or a variant thereof, for example having at least 90%, 95%, 99% or greater amino acid sequence identity thereto) whose activity (e.g., whose target binding and collateral cleavage activities) is sufficiently thermostable, for example at temperatures within a range of 60-65° C. to perform in assays as described herein (e.g., in some embodiments, one-pot assays). For example, in some embodiments, sufficient thermostable activity is activity that is reasonably comparable to (e.g., within about 25%) of an appropriate reference Thermostable Cas Protein (e.g., Aac or RS9) as described herein.

In some embodiments, the disclosure describes a detection method comprising steps of: contacting a CRISPR-Cas complex comprising: a Cas protein with collateral cleavage activity that is thermostable at temperatures above at least 60-65° C.; and a guide RNA selected or engineered to be complementary to a target nucleic acid sequence; with a sample potentially comprising a nucleic acid of the target nucleic acid sequence.

In some embodiments, the step of contacting comprises contacting the CRISPR-Cas complex and sample with a reporter susceptible to cleavage by the Cas protein collateral activity. In some embodiments, the step of contacting comprises incubating for a period of time above the temperature. In some embodiments, a detection method further comprises a step of amplifying nucleic acid present in the sample. In some embodiments, the step of amplifying utilizes a thermostable nucleic acid polymerase. In some embodiments, the steps of amplifying and contacting are performed in a single vessel.

In some embodiments, the Cas protein is a Cas12 protein. In some embodiments, the Cas protein has an amino acid sequence that is at least 80% identical to that of SEQ ID NO: 1. In some embodiments, the Cas protein has an amino acid sequence that is at least 80% identical to that of SEQ ID NO: 2. In some embodiments, the Cas protein has an amino acid sequence having at least 80% sequence identity to any one of SEQ ID Nos. 1-10. In some embodiments, the Cas protein has an amino acid sequence having 80% sequence identity to any one of SEQ ID Nos. 1-10.

In some embodiments, in a method of performing a detection assay utilizing a Cas protein with collateral cleavage activity, the improvement that comprises utilizing a Cas protein with thermostable collateral cleavage activity. In some embodiments, the Cas protein is a Cas12 protein. In some embodiments, the Cas protein has an amino acid sequence that is at least 80% identical to that of SEQ ID NO: 1. In some embodiments, the Cas protein has an amino acid sequence that is at least 80% identical to that of SEQ ID NO: 2. In some embodiments, the Cas protein has an amino acid sequence having at least 80% sequence identity to any one of SEQ ID Nos. 1-10. In some embodiments, a method of performing a detection assay is conducted in a single reaction vessel. In some embodiments, the thermostable collateral cleavage activity is thermostable above a temperature of about 60° C. In some embodiments, the thermostable collateral cleavage activity is thermostable above a temperature of about 65° C.

Those skilled in the art are aware of classification systems for Cas proteins that are used to define, for example, Cas12-type vs Cas13-type Cas proteins. In particular, those skilled in the art are familiar with sequence elements characteristic of Cas 12 vs Cas 13. See, for example, Koonin et al., Curr Opin Microbiol., 2017 June; 37:67-78, Makarova et al., Nat Rev Microbiol., 2015 November; 13 (11): 722-736, Shmakov et al., Mol Cell., 2015 Nov. 5; 60 (3): 385-397, Yan and Hunnewell et al., Science, 2018 Dec. 6, Yan et al., Mol Cell., 2018 Apr. 19; 70, 327-339, Makarova et al., Nat Rev Microbiol., 2011 June; 9 (6): 467-477, Makarova et al., CRISPR Journal, 2018, Volume 1, Number 5, Shmakov et al., Nat Rev Microbiol., 2017 March; 15 (3): 169-182, Yan and Hunnewell et al., Science, 2019 Jan. 4; 363, 88-91, Abudayyeh et al., Science, 2016 Aug. 5; 353, 6299, Gootenberg and Abudayyeh et al., Science, 2017 Apr. 28, 356, 438-442, Gootenberg and Abudayyeh et al., Science, 2018 Apr. 27; 360, 439-444.

Those skilled in the art will further appreciate that, in many embodiments, a Cas12 (e.g., a Thermostable Cas12) as provided by the present disclosure is characterized by an overall degree of sequence similarity with an exemplified Cas protein (e.g., of any of SEQ ID NO: 1-10) and/or presence of one or more sequence elements characteristic of a Cas12, a Cas13, a subspecies thereof, and/or a Thermostable Cas Protein. In some embodiments, presence of a characteristic sequence element and a particular overall sequence identity which may be reasonably low—for example 40%, 45%, 50%, 55%, 60%, 65%, 70%, 75%, 80%, 85%, 90%, or 95% of the characteristic sequence element) is indicative of a provided Cas protein as described herein. Alternatively, in some embodiments, a provided Cas protein as described herein shows high sequence identity (e.g., 90%, 91%, 92%, 93%, 94%, 95%, 96%, 97%, 98%, 99%) with an exemplified Cas (e.g., of any of SEQ ID NO:1-10), independent of presence of such characteristic sequence element. In some embodiments, both one or more characteristic sequence elements and high (e.g., 90%, 91%, 92%, 93%, 94%, 95%, 96%, 97%, 98%, 99%) sequence identity are present.

In some aspects, the present disclosure provides a detection method comprising steps of: contacting a CRISPR-Cas complex comprising: a Cas protein with collateral cleavage activity that is thermostable at temperatures above at least 60-65° C.; and a guide RNA selected or engineered to be complementary to a target nucleic acid sequence; with a sample potentially comprising a target nucleic acid sequence. In some embodiments, a contacting step comprises contacting a CRISPR-Cas complex and sample with a reporter susceptible to cleavage by a Cas protein collateral activity. In some embodiments, a contacting step comprises incubating for a period of time above the temperature.

In some embodiments, a provided detection method comprises (e.g., further comprises) a step of amplifying nucleic acid present in the sample. In some embodiments, an amplifying step may utilize a thermostable nucleic acid polymerase. In some embodiments, steps of amplifying and contacting are performed in a single vessel and/or without intervening component removal step(s) and/or wash step(s).

In some embodiments, technologies described herein utilize a Cas protein that is a Cas12 protein. In some embodiments, such a Cas protein has an amino acid sequence that is at least 80% identical to that of SEQ ID NO: 1 or SEQ ID NO: 2. In some embodiments, a utilized Cas protein has an amino acid sequence having at least 80% sequence identity to any one of SEQ ID Nos. 1-10. In some embodiments, such a Cas protein has an amino acid sequence having at least 80% sequence identity to one or more of SEQ ID Nos. 1-10.

In some aspects, the present disclosure provides improved methods of performing a detection assay utilizing a Cas protein with collateral cleavage activity, the improvement comprising utilizing a Cas protein with thermostable collateral cleavage activity. In some embodiments, a Cas protein utilized in such embodiments is a Cas12 protein. In some embodiments, a utilized Cas protein has an amino acid sequence that is at least 80% identical to that of SEQ ID NO: 1 or SEQ ID NO: 2. In some embodiments, a utilized Cas protein has an amino acid sequence having at least 80% sequence identity to one or more of SEQ ID Nos. 1-10.

In some embodiments, a provided method (e.g., an improved method) of performing a detection assay is conducted in a single reaction vessel. In some embodiments, of provided technologies, where a Cas protein with thermostable collateral cleavage activity is utilized, such activity is thermostable above a temperature of about 60° C. In some embodiments, such activity is thermostable above a temperature of about 65° C. In some embodiments, a Cas protein has an amino acid sequence having at least 80% sequence identity to one or more of SEQ ID Nos. 1-10.

In some aspects, the present disclosure provides compositions (and particularly an engineered or otherwise non-naturally occurring composition) comprising: (a) a Cas protein with collateral cleavage activity that is thermostable at temperatures above at least 60-65° C.; and (b) at least one guide capable of forming a complex with such a Thermostable Cas Protein and directing the complex to bind to a target nucleic acid sequence. In some embodiments, a utilized Cas protein has an amino acid sequence that is at least 80% identical to that of SEQ ID NO: 1 or SEQ ID NO: 2. In some embodiments, a utilized Cas protein has an amino acid sequence having at least 80% sequence identity to one or more of SEQ ID NOs: 1-10. In some embodiments, a utilized at least one guide comprises two guide sequences capable of hybridizing to two different target nucleic acid sequences or different regions of a target nucleic acid sequence. In some embodiments, a utilized at least one guide comprises a plurality of guide sequences capable of hybridizing to a plurality of different target nucleic acid sequences or a plurality of different regions of a target nucleic acid sequence. In some embodiments, a utilized guide sequence is capable of hybridizing to one or more target nucleic acid sequences in a prokaryotic cell. In some embodiments, a utilized guide sequence is capable of hybridizing to one or more target nucleic acid sequences in a eukaryotic cell.

In some aspects, the present disclosure provides compositions (and particularly an engineered or otherwise non-naturally occurring composition) comprising: (a) a polynucleotide encoding a Cas protein with collateral cleavage activity that is thermostable at temperatures above at least 60-65° C.; and at least one guide capable of forming of complex with the Cas protein and directing the complex to bind to a target nucleic acid sequence. In some embodiments, a utilized Cas protein has an amino acid sequence that is at least 80% identical to that of SEQ ID NO: 1 or SEQ ID NO: 2. In some embodiments, a utilized Cas protein has an amino acid sequence having at least 80%, sequence identity to one or more of SEQ ID NOs: 1-10. In some embodiments, a utilized at least one guide comprises two guide sequences capable of hybridizing to two different target nucleic acid sequences or different regions of a target nucleic acid sequence. In some embodiments, a utilized at least one guide comprises a plurality of guide sequences capable of hybridizing to a plurality of different target nucleic acid sequences or a plurality of different regions of a target nucleic acid sequence. In some embodiments, a utilized guide sequence is capable of hybridizing to one or more target nucleic acid sequences in a prokaryotic cell. In some embodiments, a utilized guide sequence is capable of hybridizing to one or more target nucleic acid sequences in a eukaryotic cell.

In some aspects, the present disclosure provides compositions (and particularly an engineered or otherwise non-naturally occurring composition) for modifying nucleotides in a target nucleic acid comprising a Cas protein with collateral cleavage activity that is thermostable at temperatures above at least 60-65° C. In some embodiments, a composition comprises (e.g., further comprises) at least one guide sequence capable of forming of complex with the Cas protein and directing the complex to bind to a target nucleic acid sequence. In some embodiments, a utilized Cas protein has an amino acid sequence that is at least 80% identical to that of SEQ ID NO: 1 or SEQ ID NO: 2. In some embodiments, a utilized Cas protein has an amino acid sequence having at least 80%, sequence identity to one or more of SEQ ID NOs: 1-10. In some embodiments, a utilized Cas protein has been modified to reduce off-target effects. In some embodiments, modification of the nucleotides in a target nucleic acid remedies a disease caused by a point mutation. In some embodiments, modification of nucleotides in a target nucleic acid inactivates a gene encoded by the target nucleic acid sequence. In some embodiments, modification of the nucleotides in a target nucleic acid modifies a gene product encoded by the target nucleic acid sequence. In some embodiments, modification of the nucleotides in a target nucleic acid modifies expression level of a gene product encoded by a target nucleic acid sequence. In some embodiments, a utilized at least one guide comprises two guide sequences capable of hybridizing to two different target nucleic acid sequences or different regions of a target nucleic acid sequence. In some embodiments, a utilized at least one guide comprises a plurality of guide sequences capable of hybridizing to a plurality of different target nucleic acid sequences or a plurality of different regions of a target nucleic acid sequence. In some embodiments, a utilized guide sequence is capable of hybridizing to one or more target nucleic acid sequences in a prokaryotic cell. In some embodiments, a utilized guide sequence is capable of hybridizing to one or more target nucleic acid sequences in a eukaryotic cell.

In some aspects, the present disclosure provides a vector system that comprises one or more vectors comprising: (a) a first regulatory element operably linked to a nucleotide sequence encoding a Cas protein with collateral cleavage activity that is thermostable at temperatures above at least 60-65° C.; and (b) a second regulatory element operably linked to a nucleotide sequence encoding a guide. In some embodiments, a nucleotide sequence encodes a Cas protein with an amino acid sequence that is at least 80% identical to that of SEQ ID NO: 1 or SEQ ID NO: 2. In some embodiments, a nucleotide sequence encodes a Cas protein with an amino acid sequence having at least 80% sequence identity to one or more of SEQ ID NOs: 1-10. In some embodiments, the nucleotide sequence encoding a Cas protein is codon optimized. In some embodiments, (a) and (b) are comprised in a single vector. In some embodiments, (a) and (b) are comprised in separate vectors. In some embodiments, a vector system comprises a viral vector.

In some aspects, the present disclosure provides a method of cleaving at least one target nucleic acid in a cell comprising contacting a cell with a Cas protein with collateral cleavage activity that is thermostable at temperatures above at least 60-65° C. and at least one guide capable of hybridizing to the at least one target nucleic acid, wherein the Cas protein is capable of forming a complex with the at least one guide and causing a break in the at least one target nucleic acid.

In some aspects, the present disclosure provides a method of altering expression of at least one target nucleic acid in a cell comprising contacting the cell with a Cas protein with collateral cleavage activity that is thermostable at temperatures above at least 60-65° C. and at least one guide capable of hybridizing to the at least one target nucleic acid, wherein the Cas protein is capable of forming a complex with the at least one guide and causing a break in the at least one target nucleic acid.

In some aspects, the present disclosure provides a method of altering expression of at least one target nucleic acid in a cell comprising contacting the cell with a Cas protein with collateral cleavage activity that is thermostable at temperatures above at least 60-65° C. and at least one guide capable of hybridizing to the at least one target nucleic acid, wherein the Cas protein is capable of forming a complex with the at least one guide and editing the at least one target nucleic acid sequence.

In some aspects, the present disclosure provides a method of modifying at least one target nucleic acid in a cell comprising contacting a cell with a Cas protein with collateral cleavage activity that is thermostable at temperatures above at least 60-65° C. and at least one guide capable of hybridizing to the at least one target nucleic acid, wherein the Cas protein is capable of forming a complex with the at least one guide and editing the at least one target nucleic acid sequence.

In some embodiments, editing a target nucleic acid comprises insertion of a payload nucleic acid at the target nucleic acid sequence. In some embodiments, a nucleotide sequence encodes a Cas protein with an amino acid sequence that is at least 80% identical to that of SEQ ID NO: 1 or SEQ ID NO: 2. In some embodiments, a nucleotide sequence encodes a Cas protein with an amino acid sequence having at least 80% sequence identity to one or more of SEQ ID NOs: 1-10. In some embodiments, a utilized at least one guide comprises two guide sequences capable of hybridizing to two different target nucleic acid sequences or different regions of a target nucleic acid. In some embodiments, a utilized at least one guide comprises a plurality of guide sequences capable of hybridizing to a plurality of different target nucleic acid sequences or a plurality of different regions of a target nucleic acid. In some embodiments, a utilized guide sequence is capable of hybridizing to one or more target nucleic acid sequences in a prokaryotic cell. In some embodiments, a utilized guide sequence is capable of hybridizing to one or more target nucleic acid sequences in a eukaryotic cell.

In some aspects, the present disclosure provides a nucleic acid encoding a Cas protein with collateral cleavage activity that is thermostable at temperatures above at least 60-65° C. In some embodiments, a nucleotide sequence encodes a Cas protein with an amino acid sequence that is at least 80% identical to that of SEQ ID NO: 1 or SEQ ID NO: 2. In some embodiments, a nucleotide sequence encodes a Cas protein with an amino acid sequence having at least 80% sequence identity to one or more of SEQ ID NOs: 1-10.

In some aspects, the present disclosure provides a method of treating a disorder or a disease in a subject in need thereof comprising administering to the subject a Cas protein with collateral cleavage activity that is thermostable at temperatures above at least 60-65° C. and at least one guide capable of hybridizing to a target nucleic acid. In some embodiments, a nucleotide sequence encodes a Cas protein with an amino acid sequence that is at least 80% identical to that of SEQ ID NO: 1 or SEQ ID NO: 2. In some embodiments, a nucleotide sequence encodes a Cas protein with an amino acid sequence having at least 80%, sequence identity to one or more of SEQ ID NOs: 1-10. In some embodiments, a utilized at least one guide comprises two guide sequences capable of hybridizing to two different target nucleic acid sequences or different regions of a target nucleic acid. In some embodiments, a utilized at least one guide comprises a plurality of guide sequences capable of hybridizing to a plurality of different target nucleic acid sequences or a plurality of different regions of a target nucleic acid. In some embodiments, a utilized at least one guide sequence is capable of hybridizing to one or more target nucleic acid sequences in a prokaryotic cell. In some embodiments, a utilized at least one guide sequence is capable of hybridizing to one or more target nucleic acid sequences in a eukaryotic cell. In some embodiments, a utilized Cas protein is capable of forming a complex with a guide and causing a break in a target nucleic acid. In some embodiments, a utilized Cas protein is capable of forming a complex with a guide and editing a target nucleic acid sequence.

In some aspects, the present disclosure provides compositions wherein a Cas protein is associated with a modifying entity. In some embodiments, a modifying entity is an adenosine deaminase. In some embodiments, a modifying entity is a cytidine deaminase.

In some aspects, the present disclosure provides pharmaceutical compositions comprising a Cas protein of the present disclosure.

In some aspects, the present disclosure provides a method of characterizing a Cas protein comprising assessing one or more of: (a) cis cleavage activity; (b) trans cleavage activity; (c) sensitivity; (d) preference for RNA or DNA target nucleic acid; (e) preference for RNA or DNA non-target nucleic acid; and (f) enzyme stability.

### BRIEF DESCRIPTION OF THE DRAWING

FIG. 1 demonstrates exemplary characterization of candidate Thermostable Cas Proteins, Pal1, Pal2 low MW, Pal2 high MW, and Pal3.

FIG. 2 demonstrates exemplary Pal1 and Pal2 activity at 56° C.

FIG. 3 demonstrates exemplary activity of Pal1 at 37° C., 56° C., and 70° C. with different exemplary guides.

FIG. 4 demonstrates exemplary activity of Pal1 at 56° C. and 70° C. compared to control. These data suggest activity of Pal1 is specific to target DNA.

FIG. 5 demonstrates exemplary temperature profile of Pal1.

FIG. 6 demonstrates exemplary activity of Pal2 high MW at 37° C., 56° C., and 70° C. with different exemplary guides.

FIG. 7 demonstrates exemplary activity of Pal2 high MW at 56° C. compared to control. These data suggest activity of Pal2 high MW is specific to target DNA.

FIG. 8 demonstrates exemplary temperature profile of Pal2 high MW.

FIG. 9 demonstrates an exemplary protein thermal shift assay performed following the vendor (ThermoFisher) recommended protocol. Briefly, Cas enzyme (PAL1, PAL2, PAL3, PAL4, PAL5, PAL6, PAL8, PAL9, or PAL10) (500 ng/μL) was mixed with protein thermal shift dye (8×) protein thermal shift buffer and placed in QuantStudio 5 qPCR instrument to track fluorescence changes with slowly increasing temperature. Data analysis to extract melting temperature (Tm) was done by taking the first derivative of raw fluorescence intensity in the X4-M4 channel.

FIG. 10: demonstrates collateral activity signal of thermostable PAL5 Cas12b in complex with an engineered single guide RNA (sgRNA) targeting either the N-gene or Orf1ab gene of SARS COV2. The no-target control (NTC) is shown in grey. The target is supplied as a purified in-vitro transcribed (IVT) RNA.

FIG. 11: demonstrates collateral activity signal of thermostable PAL5 Cas12b in complex with an engineered single guide RNA (sgRNA) targeting either the N-gene or Orflab gene of SARS COV2. The no-target control (NTC) is shown in grey. The target, which is supplied at low concentrations, is first amplified using LAMP, the product of which is simultaneously detected using PAL5 Cas12b.

FIG. 12: demonstrates collateral activity signal of thermostable PAL8 Cas12b in complex with an engineered single guide RNA (sgRNA) targeting the N-gene of Sars COV2. There are two variants of the sgRNA (N-1) and (N-5) that were tested which show unambiguous signal above background. The no-target control (NTC) is shown in grey. The target is supplied as a purified in-vitro transcribed (IVT) RNA.

FIG. 13: demonstrates collateral activity signal of thermostable PAL8 Cas12b in complex with an engineered single guide RNA (sgRNA) targeting the N-gene of SARS COV2 (SCOV2-N1). The no-target control (NTC) is shown in grey. The target, which is supplied at low concentrations, is first amplified using LAMP, the product of which is simultaneously detected using PAL8 Cas12b.

FIG. 14: demonstrates collateral activity signal of thermostable PAL9 Cas12b in complex with an engineered single guide-RNA (sgRNA) targeting the N-gene of SARS COV2. There are three variants of the sgRNA (N-1), (N-2), and (N-3) that were tested which show unambiguous signal above background. The no-target control (NTC) is shown in grey. The target is supplied as a purified in-vitro transcribed (IVT) RNA.

FIG. 15: demonstrates collateral activity signal of thermostable PAL9 Cas12b in complex with an engineered single guide RNA (sgRNA) targeting the N-gene of Sars COV2. The no-target control (NTC) is shown in grey. There are three variants of the sgRNA (N-1), (N-2), and (N-3) that were tested which show unambiguous signal above background. The target, which is supplied at low concentrations, is first amplified using LAMP, the product of which is simultaneously detected using PAL9 Cas12b.

FIG. 16: demonstrates collateral activity signal of thermostable PAL10 Cas12b in complex with an engineered single guide RNA (sgRNA) targeting the N-gene of SARS COV2. There are two variants of the sgRNA (N-1) and (N-3) that were tested which show unambiguous signal above background. The no-target control (NTC) is shown in grey. The target is supplied as a purified in-vitro transcribed (IVT) RNA.

FIG. 17: demonstrates collateral activity signal of thermostable PAL10 Cas12b in complex with an engineered single guide RNA (sgRNA) targeting the N-gene of Sars COV2. The no-target control (NTC) is shown in grey. There are two variants of the sgRNA (N-1) and (N-3) that were tested which show unambiguous signal above background. The target, which is supplied at low concentrations, is first amplified using LAMP, the product of which is simultaneously detected using PAL10 Cas12b.

### DEFINITIONS

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc., In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc., In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: As used herein, the term “agent”, may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in one or more certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog can be generated through performance of a synthetic process different from that used to generate the reference substance.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell). Binding between two entities may be considered “specific” if, under the conditions assessed, the relevant entities are more likely to associate with one another than with other available binding partners.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Designed: As used herein, the term “designed” refers to an agent (i) whose structure is or was selected by the hand of man; (ii) that is produced by a process requiring the hand of man; and/or (iii) that is distinct from natural substances and other known agents.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

Functional: As used herein, a “functional” biological molecule is a biological molecule in a form in which it exhibits a property and/or activity by which it is characterized. A biological molecule may have two functions (i.e., bifunctional) or many functions (i.e., multifunctional).

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Genome: As used herein, the term “genome” refers to the total genetic information carried by an individual organism or cell, represented by the complete DNA sequences of its chromosomes.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Host cell: as used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO KI, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

Human: In some embodiments, a human is an embryo, a fetus, an infant, a child, a teenager, an adult, or a senior citizen.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Intraperitoneal: The phrases “intraperitoneal administration” and “administered intraperitoneally” as used herein have their art-understood meaning referring to administration of a compound or composition into the peritoneum of a subject.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Linker: as used herein, is used to refer to that portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., chimeric systems) known in the art (see e.g., Holliger, P., et al. (1993) Proc. Natl. Acad. Sci. USA 90:6444-6448; Poljak, R. J., et al. (1994) Structure 2:1 121-1123).

Moiety: Those skilled in the art will appreciate that a “moiety” is a defined chemical group or entity with a particular structure and/or or activity, as described herein.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: as used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Oral: The phrases “oral administration” and “administered orally” as used herein have their art-understood meaning referring to administration by mouth of a compound or composition.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Payload: In general, the term “payload”, as used herein, refers to an agent that may be delivered or transported by association with another entity. In some embodiments, such association may be or include a covalent linkage; in some embodiments such association may be or include non-covalent interaction(s). In some embodiments, association may be direct; in some embodiments, association may be indirect. The term “payload” is not limited to a particular chemical identity or type; for example, in some embodiments, a payload may be or comprise, for example, an entity of any chemical class including, for example, a lipid, a metal, a nucleic acid, a polypeptide, a saccharide (e.g., a polysaccharide), small molecule, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, etc., or a combination thereof. In some embodiments, a payload may be or comprise a cell or organism, or a fraction, extract, or component thereof. In some embodiments, a payload may be or comprise a natural product in that it is found in and/or is obtained from nature; alternatively or additionally, in some embodiments, the term may be used to refer to one or more entities that is man-made in that it is designed, engineered, and/or produced through action of the hand of man and/or is not found in nature. In some embodiments, a payload may be or comprise an agent in isolated or pure form; in some embodiments, such agent may be in crude form.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide. In some embodiments, a “polypeptide” may be referred to as a “protein” (e.g., the term “Cas protein” may be used to refer to a “Cas polypeptide” as defined herein; in some embodiments, a “Cas 12 protein” may be distinguished, for example, from a “Cas 13 protein” through consideration of percent homology or identity with, and/or shared characteristic sequence element of, and appropriate reference polypeptide as described herein).

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc., In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, an appropriate reference Cas protein sequence may be found in a literature report or sequence database (e.g., GENBANK). To give but a few examples known to those skilled in the art, in some embodiments, an appropriate reference Cas12 protein or an appropriate reference Cas13 protein may be any of those described in, for example, Koonin et al., Curr Opin Microbiol., 2017 June; 37:67-78, Makarova et al., Nat Rev Microbiol., 2015 November; 13 (11): 722-736, Shmakov et al., Mol Cell., 2015 Nov. 5; 60 (3): 385-397, Yan and Hunnewell et al., Science, 2018 Dec. 6, Yan et al., Mol Cell., 2018 Apr. 19; 70, 327-339, Makarova et al., Nat Rev Microbiol., 2011 June; 9 (6): 467-477, Makarova et al., CRISPR Journal, 2018, Volume 1, Number 5, Shmakov et al., Nat Rev Microbiol., 2017 March; 15 (3): 169-182, Yan and Hunnewell et al., Science, 2019 Jan. 4; 363, 88-91, Abudayyeh et al., Science, 2016 Aug. 5; 353, 6299, Gootenberg and Abudayyeh et al., Science, 2017 Apr. 28, 356, 438-442, Gootenberg and Abudayyeh et al., Science, 2018 Apr. 27; 360, 439-444. Alternatively, in some embodiments, an exemplified Cas protein described herein (e.g., one of SEQ ID NO: 1-10) may serve as a reference relative to which other proteins are compared.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Single Nucleotide Polymorphism (SNP): As used herein, the term “single nucleotide polymorphism” or “SNP” refers to a particular base position in the genome where alternative bases are known to distinguish one allele from another. In some embodiments, one or a few SNPs and/or CNPs is/are sufficient to distinguish complex genetic variants from one another so that, for analytical purposes, one or a set of SNPs and/or CNPs may be considered to be characteristic of a particular variant, trait, cell type, individual, species, etc., or set thereof. In some embodiments, one or a set of SNPs and/or CNPs may be considered to define a particular variant, trait, cell type, individual, species, etc., or set thereof.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treatment: As used herein, the term “treatment” (also “treat” or “treating”) refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. Thus, in some embodiments, treatment may be prophylactic; in some embodiments, treatment may be therapeutic.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

### CRISPR-Cas Technologies

Typically, a Cas protein and/or a guide are the primary components of CRISPR-Cas technologies. CRISPR-Cas technologies or CRISPR-Cas systems refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide (also referred to as a “spacer” in the context of an endogenous CRISPR system), or “RNA(s)” as that term is herein used (e.g., RNA(s) to guide Cas, such as Cas proteins disclosed herein, e.g., CRISPR RNA and transactivating (tracr) RNA or a single guide RNA (sgRNA) (chimeric RNA)) or other sequences and transcripts from a CRISPR locus. In general, CRISPR-Cas technologies are characterized by elements that promote the formation of a CRISPR complex at the site of a target nucleic acid (also referred to as a protospacer in the context of an endogenous CRISPR system). In some embodiments, a tracrRNA is not required.

In some embodiments of engineered or non-naturally occurring technologies of the present disclosure, a direct repeat may encompass naturally-occurring sequences or non-naturally-occurring sequences. In some embodiments, a direct repeat (DR) may be a naturally occurring lengths and/or sequences. In some embodiments, a direct repeat can be 36 nucleotides (nt) in length, but a longer or shorter direct repeat can vary. For example, in some embodiments a direct repeat can be 30 nt or longer, such as 30-100 nt or longer. For example, a direct repeat can be 30 nt, 40 nt, 50 nt, 60 nt, 70 nt, 70 nt, 80 nt, 90 nt, 100 nt or longer in length. In some embodiments, a direct repeat of the present disclosure can include synthetic nucleotide sequences inserted between the 5′ and 3′ ends of naturally occurring direct repeats. In certain embodiments, the inserted sequence may be self-complementary, for example, 20%, 30%, 40%, 50%, 60%, 70%, 80%, 90%, or 100% self-complementary. Furthermore, a direct repeat of the present disclosure may include insertions of nucleotides such as an aptamer or sequences that bind to an adapter protein (for association with functional domains). In certain embodiments, one end of a direct repeat containing such an insertion is roughly the first half of a short DR and the end is roughly the second half of the short DR. In some embodiments, direct repeats may be identified in silico.

In some embodiments, a Cas protein is a Thermostable Cas Protein.

In the context of formation of a CRISPR complex, “target nucleic acid sequence” refers to a sequence to which a guide comprising a guide sequence has (e.g., is designed to have) complementarity, where hybridization between a target nucleic acid sequence and a guide sequence promotes the formation of a CRISPR complex. In some embodiments, a target nucleic acid comprises any polynucleotide, such as DNA or RNA polynucleotides. In some embodiments, a target nucleic acid is located in the nucleus or cytoplasm of a cell. In some embodiments, a target nucleic acid is ex vivo. In some embodiments, a target nucleic acid is present in an in vitro system. In some embodiments, a target nucleic acid is present in a sample, e.g., in a biological sample or in an environmental sample.

In some embodiments, a guide (e.g., constant domain and/or spacer) is about or more than about 5, 10, 11, 12, 13, 14, 15, 16, 17, 18, 19, 20, 21, 22, 23, 24, 25, 26, 27, 28, 29, 30, 35, 40, 45, 50, 75, 100, 125, 150, 160, 170, 180, 190 or more nucleotides in length. In some embodiments, a guide is less than about 200, 190, 180, 170, 160, 150, 140, 130, 120, 110, 100, 90, 80, 75, 50, 45, 40, 35, 30, 25, 20, 15, 12, or fewer nucleotides in length. In some embodiments, the guide is 10-30 nucleotides long, such as 20-30 or 20-40 nucleotides long or longer, such as 30 nucleotides long or about 30 nucleotides long for CRISPR-Cas effectors. In certain embodiments, a guide is 10-30 nucleotides long, such as 20-30 nucleotides long, such as 30 nucleotides long. In certain embodiments, a guide is 90-200 nucleotides long, such as 100-190 nucleotides long, such as 110-180 nucleotides long, such as 120-170 nucleotides long. The ability of a guide to direct sequence-specific binding of a CRISPR complex to a target nucleic acid may be assessed by any suitable assay. For example, in some embodiments, the components of a CRISPR system sufficient to form a CRISPR complex, including the guide sequence to be tested, are provided to a host cell having the corresponding target nucleic acid sequence, such as by transfection with vectors or vector systems encoding the components of CRISPR-Cas technologies, as discussed elsewhere herein, followed by an assessment of preferential cleavage within the target nucleic acid sequence. Similarly, in some embodiments, cleavage of a target nucleic acid is evaluated, for example, in a test tube by providing the target nucleic acid, components of a CRISPR complex, including the guide sequence to be tested and a control guide sequence different from the test guide sequence, and comparing binding or rate of cleavage at the target nucleic acid sequence between the test and control guide sequence reactions. Other assays are possible, and will occur to those skilled in the art.

In some embodiments, degree of complementarity between a guide sequence and its corresponding target nucleic acid sequence can be about or more than about 50%, 60%, 75%, 80%, 85%, 90%, 95%, 97.5%, 99%, or 100%; in some embodiments, a guide or RNA or crRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or, in some embodiments, guide or RNA or crRNA can be less than about 75, 50, 45, 40, 35, 30, 25, 20, 15, 12, or fewer nucleotides in length; and advantageously tracr RNA is 30 or 50 nucleotides in length. In some embodiments, degree of complementarity between a guide sequence and its corresponding target nucleic acid sequence is greater than 94.5% or 95% or 95.5% or 96% or 96.5% or 97% or 97.5% or 98% or 98.5% or 99% or 99.5% or 99.9%, or 100%. In some embodiments, off target is less than 100% or 99.9% or 99.5% or 99% or 99% or 98.5% or 98% or 97.5% or 97% of 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the target nucleic acid sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the target nucleic acid sequence and the guide.

In some embodiments, modulations of cleavage activity (e.g., efficiency) can be exploited by introduction of mismatches, e.g., 1 or more mismatches, such as 1 or 2 mismatches between a guide sequence and target nucleic acid sequence, including the position of the mismatch along the guide sequence/target nucleic acid sequence. Without wishing to be bound by any one theory, in some embodiments, by choosing mismatch position along the guide sequence, cleavage activity (e.g., efficiency) can be modulated. For example, in some embodiments, if less than 100% cleavage of targets is desired (e.g., in a cell population), 1 or more, such as preferably 2 mismatches between a guide sequence and a target nucleic acid sequence may be introduced in the guide sequence. In some embodiments, the more central along the guide sequence of the mismatch position, the lower the cleavage activity (e.g.,

In some embodiments, formation of a CRISPR complex (comprising a guide sequence hybridized to a target nucleic acid sequence and complexed with one or more Cas proteins) results in cleavage in or near (e.g., within 1, 2, 3, 4, 5, 6, 7, 8, 9, 10, 20, 50, or more base pairs from) the target nucleic acid sequence. Without wishing to be bound by any one theory, cleavage site location in or near a target nucleic acid sequence may depend on, for example, secondary structure, in particular in the case of RNA targets. In some cases, in the context of an endogenous CRISPR system, formation of a CRISPR complex (comprising a guide sequence hybridized to a target nucleic acid sequence and complexed with one or more Cas proteins) results in cleavage of one or both strands (if applicable) in or near (e.g., within 1, 2, 3, 4, 5, 6, 7, 8, 9, 10, 20, 50, or more base pairs from) the target nucleic acid sequence.

### Thermostable Cas Proteins

As described herein, the present disclosure identifies the source of a problem with certain Cas proteins, for example Cas proteins with collateral activity, as described above, in that certain such proteins activity are insufficiently stable at elevated temperatures. This can prove particularly challenging, for example, when utilizing Cas proteins in systems or assays that benefit from or require elevated temperatures (e.g., at temperatures at which nucleic acid extension and/or amplification are performed, and/or in cells or organisms (e.g., thermophilic organisms that survive or thrive only at elevated temperatures). Additionally, the present disclosure further surprisingly demonstrates that, for some proteins, loss of activity upon temperature elevation may be irreversible. This reality increases the significance of the insight, provided by the present disclosure, that Cas proteins with thermostable activity (e.g., “Thermostable Cas Proteins”), specifically including Cas proteins with thermostable collateral activity, are particularly desirable.

The present disclosure therefore provides improved Cas proteins, and particularly provides improved Cas proteins with collateral activity, and furthermore provides improved technologies that utilize a Thermostable Cas Protein (e.g., whose collateral activity is thermostable) as described herein.

In some embodiments, the present disclosure provides non-naturally occurring or engineered compositions for binding to, detecting, and/or modifying nucleotides in a target nucleic acid comprising a Thermostable Cas Protein as described herein (e.g., Cas proteins with collateral cleavage activity that is thermostable at temperatures above at least 60-65° C.). In some such embodiments, provided compositions comprise non-naturally occurring or engineered compositions comprising a Thermostable Cas Protein as described herein (e.g., Cas proteins with collateral cleavage activity that is thermostable at temperatures above at least 60-65° C.) and at least one guide capable of forming a complex with the Thermostable Cas Protein and directing the complex to bind to a target nucleic acid. In some embodiments, a provided composition comprises a Cas protein/guide complex. In some embodiments, a provided composition comprises a Cas protein/guide complex bound to a target nucleic acid. In some embodiments, a provided composition comprises a Cas protein combined with a nucleic acid susceptible to collateral cleavage; in some such embodiments, such susceptible nucleic acid does not include a guide binding site and/or is not linked to and/or otherwise associated with a target nucleic acid that does include such a guide binding site. In some embodiments, a susceptible nucleic acid is labeled so that its cleavage (e.g., via collateral activity of a Cas protein as provided herein) is detectable (e.g., by release of fluorescence or visible color).

In some embodiments, the present disclosure provides non-naturally occurring or engineered compositions comprising a polynucleotide encoding a Thermostable Cas Protein as described herein (e.g., Cas proteins with collateral cleavage activity that is thermostable at temperatures above at least 60-65° C.); in some such embodiments, such composition may be combined with (e.g., used in combination with and at least one guide capable of forming of complex with the Cas protein and directing the complex to bind to a target nucleic acid sequence.

In some embodiments, a Cas enzyme provided herein (e.g., with thermostable collateral cleavage activity) is a homolog (e.g., ortholog) of a Cas enzyme that either does not have demonstrable collateral cleavage activity, or has demonstrable collateral cleavage activity but loses such activity above a relevant temperature as described herein.

In some embodiments, a Cas enzyme with thermostable collateral cleavage activity as described herein is a Cas12 (e.g., Cas12a or Cas12b) enzyme. In some embodiments, a Cas enzyme with thermostable collateral cleavage activity as described herein is a Cas enzyme comprising an amino acid sequence having 80%, 85%, 90%, 99% or 100% sequence identity to any one of SEQ ID Nos. 1-10. In some embodiments, improved collateral activity assays as described herein are performed using a Cas enzyme comprising an amino acid sequence having 80%, 85%, 90%, 99% or 100% sequence identity to any one of SEQ ID Nos. 1-10.

### Codon Optimization

Without wishing to be bound by any one theory, some species exhibit codon bias (i.e., differences in codon usage by organism) which can correlate with the efficiency of translation of mRNA by utilizing codons in mRNA that correspond with the abundance of tRNA species for that codon in a particular organism. Numerous methods are known in the art for codon optimization. In some embodiments, codons are optimized by computational methods.

In some embodiments, codon optimization refers to modification of nucleic acid sequences for enhanced expression in cells by replacing at least one codon (e.g., 1, 2, 3, 4, 5, 6, 7, 8, 9, 10, 15, 20, 25, 30, 35, 40, 45, 50, or more codons) relative to an appropriate reference sequence (e.g., a native sequence). In some embodiments, codons of an appropriate reference sequence are replaced with codons that are more frequently used or most frequently used in genes of the cell while maintaining the native amino acid sequence encoded by the nucleic acid sequence.

In some embodiments, nucleotide sequences encoding Cas proteins of the present disclosure are codon optimized. In some embodiments, nucleotide sequences encoding Cas proteins of the present disclosure are codon optimized for expression in eukaryotic cells. In some such embodiments, eukaryotic cells are human cells. In some embodiments, nucleotide sequences encoding Cas proteins of the present disclosure are codon optimized for expression in prokaryotic cells.

### Modifying Entities

In some embodiments, Cas proteins of the present disclosure are associated (e.g., fused i.e., covalently linked or non-covalently bound) to one or more modifying entities, as chimeric systems, that can modify (e.g., edit) nucleic acid sequences (e.g., target nucleic acid sequences) and/or nucleic acid (e.g., target nucleic acid) structure, for example by chemically modifying nucleotide bases.

In some embodiments, modifying entities have base editing activity. In some embodiments, a modifying entity is a deaminase. In some such embodiments, a modifying entity is a cytidine deaminase or functional fragment thereof. In some embodiments, a cytidine deaminase or functional fragment thereof comprises a sequence (i.e., nucleotide or amino acid) identity of 70%, 75%, 80%, 85%, 90%, 91%, 92%, 93%, 94%, 95%, 96%, 97% 98%, 99% or more with any cytidine deaminase known in the art. In some embodiments, a cytidine deaminase or functional fragment thereof demonstrates a cytidine deaminase activity (e.g., converting C to U). In some such embodiments, a modifying entity is an adenosine deaminase or functional fragment thereof. In some embodiments, an adenosine deaminase or functional fragment thereof comprises a sequence (i.e., nucleotide or amino acid) identity of 70%, 75%, 80%, 85%, 90%, 91%, 92%, 93%, 94%, 95%, 96%, 97% 98%, 99% or more to any adenosine deaminase known in the art. In some embodiments, an adenosine deaminase or functional fragment thereof demonstrates an adenosine deaminase activity (e.g., converting A to I).

In some embodiments, modifying entities modify target nucleic acid sequences in a site-specific manner. In some embodiments, for example, modifying entity activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, a chimeric system of the present disclosure comprises one modifying entity. In some embodiments, a chimeric system comprises a plurality of modifying entities (e.g., at least two modifying entities). In some embodiments, a chimeric system comprises a modifying entity C-terminal to a Cas protein. In some embodiments, a chimeric system comprises a modifying entity N-terminal to a Cas protein. In some embodiments, a modifying entity and a Cas protein of a chimeric system are directly linked. In some embodiments, a modifying entity and a Cas protein of a chimeric system are indirectly linked (e.g., by a linker).

Exemplary characterization of Thermostable Cas Proteins

Among other things, the present disclosure provides methods of characterizing Cas Proteins (e.g., Thermostable Cas Proteins of the present disclosure). In some embodiments, Cas proteins are characterized for one or more of cis (e.g., target nucleic acid) cleavage activity, trans or “collateral” (e.g., non-target nucleic acid) cleavage activity, sensitivity, preference for an RNA and/or DNA target nucleic acid, preference for an RNA and/or DNA non-target nucleic acid, and/or enzyme stability.

In some embodiments, Cas proteins are characterized with one guide. In some embodiments, Cas proteins are characterized with more than one guide (e.g., 2, 3, 4, 5, 6, 7, 8, 9, 10, 25, 50, 75, 100, 250, 500, 750, 1,000, or more guide sequences). In some such embodiments, the more than one guides comprise different guide sequences.

In some embodiments, Cas proteins are characterized at a particular temperature. In some such embodiments, a particular temperature comprises 25° C., 30° C., 35° C., 37° C., 40° C., 42° C., 45° C., 47° C., 50° C., 52° C., 54° C., 55° C., 56° C., 57° C., 58° C., 60° C., 62° C., 65° C., 67° C., 70° C., 72° C., 80° C., 90° C., or 100° C. In some embodiments, Cas proteins are characterized over a range of temperatures. In some such embodiments, a range of temperatures comprises 10-100° C., 20-90° C., 30-80° C., 40-70° C., 50-70° C., 10-90° C., 10-80° C., 10-70° C., 20-100° C., 20-80° C., 20-70° C., 30-100° C., 30-90° C., or 30-70° C. In some embodiments, Cas proteins are characterized at a plurality of temperatures. In some such embodiments, a plurality of temperatures comprises any combination of any temperature between 10-100° C.

### Cis Cleavage Activity

In some embodiments, Thermostable Cas Proteins as described herein are characterized for cis (e.g., target nucleic acid) cleavage activity. In some embodiments, cis cleavage activity is characterized by an in vitro cleavage assay. In some such embodiments, an in vitro cleavage assay comprises, for example, expressing a Cas protein in host cells, preparing a cell lysate, preparing (e.g., amplifying) target nucleic acids (e.g., DNA and/or RNA), incubating the Cas containing cell lysate with target nucleic acid and a guide, and assessing cleavage activity (e.g., by gel electrophoresis, reporter) compared to an appropriate reference standard. In some embodiments, an appropriate reference standard is host cells expressing a control protein that does not demonstrate cis cleavage activity (e.g., green fluorescent protein).

In some embodiments, cis cleavage activity is characterized by an ex vivo cleavage assay. In some such embodiments, an ex vivo cleavage assay comprises, for example, expressing a Cas protein in host cells and a guide, extracting polynucleotides of interest from the host cells (e.g., DNA and/or RNA), and sequencing to determine cleavage activity (e.g., insertion, deletion, and/or mutation patterns). In some embodiments, sequencing comprises deep sequencing. In some embodiments, sequencing comprises next-generation sequencing.

In some embodiments, cis cleavage activity is characterized by an endpoint assay. In some embodiments, cis cleavage activity is characterized by a kinetic assay.

### Trans Cleavage Activity

In some embodiments, Cas proteins as described herein are characterized for trans or “collateral” (e.g., non-target nucleic acid) cleavage activity. Trans or “collateral” activity is non-specific cleavage activity of non-target nucleic acids after a Cas protein binds a target nucleic acid (e.g., is an “activated” Cas protein). In some embodiments, upon recognition of a target nucleic acid, a Cas protein's trans cleavage activity is activated, so that it cleaves non-target nucleic acid (DNA or RNA or both, depending on the enzyme). In some embodiments, trans cleavage activity is assessed by a reporter. In some such embodiments, a reporter comprises the relevant cleavable nucleic acid (e.g., DNA or RNA), appropriately configured (e.g., labeled) so that its cleavage as a result of the activated collateral activity is detectable (e.g., separates a fluorophore from a quencher so that fluorescence becomes detectable, etc.). In some embodiments, a negative control (e.g., no target nucleic acid control) is used. In some such embodiments, trans cleavage activity is assessed in vitro.

In some embodiments, trans cleavage activity is characterized by an endpoint assay. In some embodiments, trans cleavage activity is characterized by a kinetic assay.

### Sensitivity

In some embodiments, Cas proteins as described herein are characterized for sensitivity. In some embodiments, sensitivity is assessed by evaluating the lowest concentration of target nucleic acid that can be detected, cleaved, and/or modified 80%, 85%, 90%, 95%, 99%, or more of the time. In some such embodiments, sensitivity is determined by assessing detection, cleavage, and/or modification of a target nucleic acid by contacting a Cas enzyme with dilutions (e.g., serial dilutions) of target nucleic acid.

Preference for a RNA or DNA Target Nucleic Acid and/or Non-Target Nucleic Acid

In some embodiments, Cas proteins as described herein are characterized for preference of RNA or DNA target nucleic acids (e.g., cis cleavage activity) and/or non-target nucleic acids (e.g., trans or “collateral” cleavage activity).

In some embodiments, Cas proteins as described herein are characterized for preference of RNA or DNA target nucleic acids (e.g., cis cleavage activity). In some such embodiments, preference is characterized by assessing cis Cas protein activity by comparing cleavage activity when contacting a Cas protein with a DNA target nucleic acid compared to an RNA target nucleic acid. In some embodiments, a DNA target nucleic acid is a double-stranded DNA. In some embodiments, a DNA target nucleic acid is a single-stranded DNA.

In some embodiments, Cas proteins as described herein are characterized for preference of RNA or DNA non-target nucleic acids (e.g., trans or “collateral” cleavage activity). In some such embodiments, preference is characterized by assessing trans Cas protein activity by comparing trans cleavage activity when contacting a Cas protein with a DNA non-target nucleic acid compared to an RNA non-target nucleic acid. In some embodiments, a DNA non-target nucleic acid is a double-stranded DNA. In some embodiments, a DNA non-target nucleic acid is a single-stranded DNA. In some embodiments, a non-target nucleic acid comprises a reporter (e.g., a reporter wherein cleavage separates a fluorophore from a quencher so that fluorescence becomes detectable, etc.). In some such embodiments, a reporter is DnaseAlert. In some such embodiments, a reporter is RnaseAlert.

### Enzyme Stability

In some embodiments, Thermostable Cas Proteins as described herein are characterized for enzyme stability. In some such embodiments, enzyme stability is characterized by evaluating enzyme denaturation (e.g., using a protein melting method). In some embodiments, evaluating enzyme denaturation comprises mixing Cas protein with buffer and dye and running a melt curve. Without wishing to be bound by any one theory, in some embodiments, as temperature increases Cas proteins unfold exposing hydrophobic regions that the dye can bind. Upon dye binding, the dye fluoresces. In some such embodiments, change in fluorescence over change in temperature is plotted against the temperatures of the melt curve. In some embodiments, melting temperature of the Cas protein can be determined and compared to that of an appropriate reference standard (e.g., Cas proteins with a known thermostability, e.g., Aac and/or RS9). In some such embodiments, changes in melting temperature are correlated to changes in protein stability (e.g., thermostability) and activity.

### Target Nucleic Acid

In some embodiments, a useful target nucleic acid in accordance with the present disclosure is not limited to a particular length; in some embodiments, a target nucleic acid is a nucleotide of any length (oligonucleotides or polynucleotides) comprising a sequence to which a guide sequence hybridizes. In some embodiments, a target nucleic comprises a three dimension structure. In some embodiments, a target nucleic acid sequence comprises coding and/or non-coding regions. In some embodiments, a target nucleic acid sequence comprises exons, introns, mRNA, tRNA, rRNA, siRNA, shRNA, miRNA, ribozymes, cDNA, plasmids, vectors, exogenous nucleotide sequences, and/or endogenous nucleotide sequences. In some embodiments, a target nucleic acid sequence comprises modified nucleotides, for example, including methylated nucleotides or nucleotide analogs. In some embodiments, a target nucleic acid sequence may be interspersed with non-nucleic acid components. In some embodiments, a target nucleic acid is a single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

In some embodiments, a target nucleic acid is recognized by CRISPR-Cas technologies and binds a Cas protein as described herein. In some embodiments, a target nucleic acid is modified or cleaved or a gene encoded by a target nucleic acid has altered expression as a result of Cas protein binding and activity. In some embodiments, a target nucleic acid sequence comprises a specific, recognizable, protospacer adjacent motif (PAM).

### Guides

In some embodiments of the present disclosure, CRISPR-Cas technologies comprises at least one guide comprising a guide sequence. In some embodiments, a guide sequence is a nucleotide sequence with sufficient complementarity that a guide is capable of hybridizing to a specific target nucleic acid. In some embodiments, a guide comprises a guide sequence that is complementary to a target nucleic acid sequence. In some embodiments, a guide sequence is 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more complementary to a target nucleic acid sequence. In some embodiments, a guide sequence is 5, 6, 7, 8, 9, 10, 11, 12, 13, 14, 15, 16, 17, 18, 19, 20, 21, 22, 23, 24, 25, or more nucleotides in length.

In some embodiments, technologies of the present disclosure utilize a plurality of guides. In some embodiments, a plurality of guides comprises 2, 3, 4, 5, 6, 7, 8, 9, 10, 20, 30, 40, 50, 60, 70, 80, 90, 100, 250, 500, 750, 1,000 or more guides. In some embodiments, two or more guide comprise the same guide sequence. In some embodiments, two more guides comprise different guide sequences. In some embodiments, two or more guide sequences hybridize to different target sites of the same target nucleic acid. In some embodiments, two or more guide sequences hybridize to different target nucleic acid sequences.

In some embodiments, the capability of a guide to direct sequence-specific binding of CRISPR-Cas technologies to a target nucleic acid may be assessed by any suitable assay. For example, the components of CRISPR-Cas technologies disclosed herein are sufficient to form a CRISPR complex, including the guide sequence to be tested, may be provided to a cell having the corresponding (e.g., complementary) target nucleic acid sequence, such as by transfection with vectors encoding components of the CRISPR-Cas technologies (e.g., as described elsewhere herein), followed by a characterization of preferential cleavage within the target nucleic acid sequence. Similarly, cleavage of a target nucleic acid may be evaluated, for example, in a test tube by providing a target nucleic acid, components of CRISPR-Cas technologies, including the guides to be tested and a control guides comprising guide sequences different from the test guide sequence, and comparing binding or rate of cleavage at the target nucleic acid between the test and control guide sequence reactions. Other assays are possible, and will occur to those skilled in the art. A guide sequence may be selected to target any target nucleic acid sequence. In some embodiments, the target nucleic acid sequence is a nucleic acid sequence within a genome of a cell. Exemplary target nucleic acids include those that are unique in the target genome.

In some embodiments, a composition comprises a Cas protein and a heterologous guide sequence, e.g., a guide sequence and the Cas protein does not exist in the same cell or the same species in nature.

In some embodiments, CRISPR-Cas technologies as described herein use a crRNA or analogous polynucleotide comprising a guide sequence, wherein the polynucleotide is an RNA, a DNA or a mixture of RNA and DNA, and/or wherein the polynucleotide comprises one or more nucleotide analogs. In some such embodiments, the sequence can comprise any structure, including but not limited to a structure of a native crRNA, such as a bulge, a hairpin or a stem loop structure. In some embodiments, the polynucleotide comprising the guide sequence forms a duplex with a second polynucleotide sequence which can be an RNA or a DNA sequence.

In some embodiments, guides of the present disclosure comprise non-naturally occurring nucleic acids and/or non-naturally occurring nucleotides, and/or nucleotide analogs, and/or chemically modifications. Non-naturally occurring nucleic acids can include, for example, mixtures of naturally and non-naturally occurring nucleotides. In some embodiments, non-naturally occurring nucleotides and/or nucleotide analogs are modified at the ribose, phosphate, and/or base moiety. In some embodiments, a guide comprises ribonucleotides and non-ribonucleotides. In some such embodiments, a guide comprises one or more ribonucleotides and one or more deoxyribonucleotides. In some embodiments, a guide comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, or bridged nucleic acids (BNA). Other examples of modified nucleotides include, but are not limited to, 2′-0-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (Y), Nl-methylpseudouridine (me 1 Y), 5-methoxyuridine (5moU), inosine, 7-methylguanosine. Examples of guide RNA chemical modifications include, without limitation, incorporation of 2′-0-methyl (M), 2′-0-methyl 3′ phosphorothioate (MS), S-constrained ethyl (cEt), or 2′-0-methyl 3′thioPACE (MSP) at one or more terminal nucleotides. In some embodiments, such chemically modified guide RNAs can comprise increased stability and increased activity as compared to unmodified guide RNAs, though on-target vs. off-target specificity is not predictable. (See, Hendel, 2015, Nat Biotechnol. 33 (9): 985-9, doi: 10.1038/nbt.3290, published online 29 Jun. 2015; Allerson et ah, J. Med. Chem. 2005, 48:901-904; Bramsen et ah, Front. Genet., 2012, 3:154; Deng et al., PNAS, 2015, 112:11870-11875; Sharma et ah, MedChemComm., 2014, 5:1454-1471; Li et al., Nature Biomedical Engineering, 2017, 1, 0066 DOI: 10.1038/s41551-017-0066).

In some embodiments, the 5’ and/or 3′ end of a guide is modified by a variety of functional moieties including fluorescent dyes, polyethylene glycol, cholesterol, proteins, or detection tags. (See Kelly et al., 2016, J. Biotech. 233:74-83). In some embodiments, a guide comprises ribonucleotides in a region that binds to a target nucleic acid and one or more deoxyribonucleotides and/or nucleotide analogs in a region that binds to a Cas protein. In some embodiments, deoxyribonucleotides and/or nucleotide analogs are incorporated in guide sequences, such as, without limitation, 5′ and/or 3′ end, stem-loop regions, and the seed region. In some embodiments, the modification is not in the 5′-handle of the stem-loop regions. In some embodiments, chemical modification in the 5′-handle of the stem-loop region of a guide may abolish its function (see Li, et al., Nature Biomedical Engineering, 2017, 1:0066). In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75 or more nucleotides of a guide sequence are chemically modified. In some embodiments, 3-5 nucleotides at either the 3′ or the 5′ end of a guide are chemically modified. In some embodiments, only minor modifications are introduced in the seed region, such as 2′-F modifications. In some embodiments, 2′-F modification is introduced at the 3′ end of a guide. In some embodiments, three to five nucleotides at the 5′ and/or the 3′ end of the guide are chemically modified with T-O-methyl (M), 2′-0-methyl-3′-phosphorothioate (MS), S-constrained ethyl (cEt), or 2′-0-methyl-3′-thioPACE (MSP). In some embodiments, such modifications can enhance genome editing efficiency (see Hendel et al., Nat. Biotechnol. (2015) 33 (9): 985-989). In some embodiments, all of the phosphodiester bonds of a guide are substituted with phosphorothioates (PS) for enhancing levels of gene disruption. In some embodiments, more than five nucleotides at the 5′ and/or the 3′ end of the guide are chemically modified with 2′-OMe, 2′-F or S-constrained ethyl (cEt). In some embodiments, such chemically modified guides can mediate enhanced levels of gene disruption (see Ragdarm et al., 0215, PNAS, E7110-E7111). In some embodiments, a guide is modified to comprise a chemical moiety at its 3′ and/or 5′ end. In some embodiments, such moieties include, but are not limited to amine, azide, alkyne, thio, dibenzocyclooctyne (DBCO), or Rhodamine. In some embodiments, the chemical moiety is conjugated to a guide sequence by a linker, such as an alkyl chain. In some embodiments, the chemical moiety of a modified guide can be used to attach the guide to another molecule, such as DNA, RNA, protein, or nanoparticles. In some embodiments, such chemically modified guides can be used, for example, to identify or enrich cells generically edited by a CRISPR system (see Lee et al., eLife, 2017, 6: e25312, DOI: 10.7554).

In some embodiments, the modification to the guide is a chemical modification, an insertion, a deletion or a split. In some embodiments, the chemical modification includes, but is not limited to, incorporation of 2′-0-methyl (M) analogs, 2′-deoxy analogs, 2-thiouridine analogs, N6-methyladenosine analogs, 2′-fluoro analogs, 2-aminopurine, 5-bromo-uridine, pseudouridine (Y), Nl-methylpseudouridine (me 1 Y), 5-methoxyuridine (5moU), inosine, 7-methylguanosine, 2′-Omethyl-3′-phosphorothioate (MS), S-constrained ethyl (cEt), phosphorothioate (PS), or 2′-0-methyl-3′-thioPACE (MSP). In some embodiments, a guide comprises one or more of phosphorothioate modifications. In some embodiments, at least 1, 2, 3, 4, 5, 6, 7, 8, 9, 10, 11, 12, 13, 14, 15, 16, 17, 18, 19, 20, or 25 nucleotides of a guide are chemically modified. In some embodiments, one or more nucleotides in the seed region are chemically modified. In some embodiments, one or more nucleotides in the 3′-terminus are chemically modified. In some embodiments, none of the nucleotides in the 5′-handle is chemically modified. In some embodiments, the chemical modification in the seed region is a minor modification, such as incorporation of a 2′-fluoro analog. In some embodiments, such chemical modifications at the 3′-terminus of the Cpf1 CrRNA improve gene cutting efficiency (see Li, et al., Nature Biomedical Engineering, 2017, 1:0066).

In some embodiments, the loop of the 5′-handle of a guide is modified. In some embodiments, the loop of the 5′-handle of a guide is modified to have a deletion, an insertion, a split, or chemical modifications. In some embodiments, the loop comprises 3, 4, or 5 nucleotides.

In some embodiments, a guide comprises portions that are chemically linked or conjugated via a non-phosphodiester bond. In some embodiments, a guide sequence comprises, in non-limiting examples, direct repeat sequence portion and a targeting sequence portion that are chemically linked or conjugated via a non-nucleotide loop. In some embodiments, the portions are joined via a non-phosphodiester covalent linker. Examples of the covalent linker include but are not limited to a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, portions of guides are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, the non-targeting guide portions can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. In some embodiments, once a non-targeting portions of a guide is functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, one or more portions of a guide can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

In some embodiments, guide portions can be covalently linked using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8:570-9; Behlke et al., Oligonucleotides (2008) 18:305-19; Watts, etak, Drug. Discov. Today (2008) 13:842-55; Shukla, et al., ChemMedChem (2010) 5:328-49.

In some embodiments, guide portions can be covalently linked using click chemistry. In some embodiments, guide portions can be covalently linked using a triazole linker. In some embodiments, guide portions can be covalently linked using Huisgen 1,3-dipolar cycloaddition reaction involving an alkyne and azide to yield a highly stable triazole linker (He et al., ChemBioChem (2015) 17:1809-1812; WO 2016/186745). In some embodiments, guide portions are covalently linked by ligating a 5′-hexyne portion and a 3′-azide portion. In some embodiments, either or both of the 5′-hexyne guide portion and a 3′-azide guide portion can be protected with 2′-acetoxyethl orthoester (2′-ACE) group, which can be subsequently removed using Dharmacon protocol (Scaringe et al., J. Am. Chem. Soc. (1998) 120:11820-11821; Scaringe, Methods Enzymol. (2000) 317:3-18).

In some embodiments, guide portions can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of ethylene and propylene glycols), polyamines group (e.g., spennine, spermidine and polymeric analogs thereof), polyesters (e.g., poly(ethyl acrylate)), polyphosphodiesters, alkylenes, and combinations thereof. In some embodiments, suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

In some embodiments, a useful linker (e.g., a non-nucleotide loop) in accordance with the present disclosure is not limited to a particular length; in some embodiments, a useful linker is a linker of any length. In some embodiments, the linker has a length equivalent to about 0-16 nucleotides. In some embodiments, the linker has a length equivalent to about 0-8 nucleotides. In some embodiments, the linker has a length equivalent to about 0-4 nucleotides. In some embodiments, the linker has a length equivalent to about 2 nucleotides. Example linker design is also described in WO2011/008730.

### Multiplexing

In some embodiments, CRISPR-Cas technologies described herein utilize a plurality of guides. Without wishing to be bound by any one theory, use of a plurality of guides enables targeting of a plurality of target nucleic acids. In some embodiments, the plurality of guides are tandemly arranged and can be optionally separated by a nucleic acid sequence (e.g., a DR sequence). In some embodiments, the position of the more than one guides in the tandem does not influence activity.

In some embodiments, CRISPR-Cas technologies as described herein utilize a plurality of guides for multiplexing. In some embodiments, more than one Cas protein is used. In some embodiments, a single Cas protein is used. In some such embodiments, a single Cas protein is delivered with a plurality of guides, e.g., at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 150, at least 200, at least 250, at least 300, at least 350, at least 400, at least 450, at least 500, at least 750, at least 1,000 or more guides.

In some embodiments, a guide or a plurality of guides hybridize to a plurality of target nucleic acids. In some embodiments, CRISPR-Cas technologies cleave and/or edit a plurality target nucleic acids. In some such embodiments, cleavage and/or editing mutates, inserts, and/or deletes nucleotides of a target nucleic acid or a plurality of target nucleic acids. In some embodiments, mutation, insertion, and/or deletion of nucleotides results in alteration of gene expression of a gene encoded by a target nucleic acid or controlled by a regulatory element of a target nucleic acid.

In some embodiments, a plurality of guide sequences are capable of hybridizing to a plurality of different target nucleic acids or different regions (e.g., sequences) of the same target nucleic acid. In some embodiments, use of CRISPR-Cas technologies with a plurality of guides as described herein provides methods for altering expression of a plurality of gene products. In some embodiments, the method comprises contacting a cell with a Cas protein with collateral cleavage activity that is thermostable at temperatures above at least 60-65° C. and at least one guide sequence capable of hybridizing to a least one target nucleic acid, wherein the Cas protein is capable of forming a complex with the at least one guide and causing a break in the at least one target nucleic acid. In some embodiments, the method comprises contacting a cell with a Cas protein with collateral cleavage activity that is thermostable at temperatures above at least 60-65° C. and at least one guide capable of hybridizing to at least one target nucleic acid, wherein the Cas protein is capable of forming a complex with the at least one guide and editing the at least one target nucleic acid sequence.

### Methods of Modifying Target Nucleic Acid Sequences

In some embodiments, CRISPR-Cas technologies described herein can be used for modifying target nucleic acid sequences (e.g., gene editing). In some embodiments, modifying target nucleic acid sequences can result in gene silencing or an alteration in the expression level (e.g., an increase or decrease) in the expression of a gene product regulated or encoded by a target nucleic acid sequence. In some embodiments, CRISPR-Cas technologies of the present disclosure can be used for site-specific modification of a target nucleic acid sequence. In some embodiments, a site-specific modification of a target nucleic acid sequence results in gene silencing or an alteration in the expression level (e.g., an increase or decrease) in the expression of a gene product regulated or encoded by a target nucleic acid sequence. Accordingly, in some embodiments, CRISPR-Cas technologies as described herein are used in methods of modifying target nucleic acid sequences. In some embodiments, CRISPR-Cas technologies as described herein are used in a method of modifying a target nucleic acid in a cell(s) (e.g., prokaryotic or eukaryotic cells).

In some embodiments, methods of the present disclosure comprehends inducing one or more nucleotide modifications in a cell comprising delivering to cell a vector or vector system as discussed elsewhere herein. In some embodiments, mutation(s) include the introduction, deletion, or substitution of one or more nucleotides or payloads at each target nucleic acid sequence of cell(s) via the guide(s), RNA(s), or sgRNA(s). In some embodiments, mutation(s) include the introduction, deletion, or substitution of 1-75 nucleotides at each target nucleic acid sequence of said cell(s) via the guide(s). In some embodiments, mutation(s) include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target nucleic acid sequence of said cell(s) via the guide(s). In some embodiments, mutation(s) include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target nucleic acid sequence of said cell(s) via the guide(s). In some embodiments, mutation(s) include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target nucleic acid sequence of said cell(s) via the guide(s). In some embodiments, mutation(s) include the introduction, deletion, or substitution of 20, 21, 22, 23, 24, 25, 26, 27, 28, 29, 30, 35, 40, 45, 50, or 75 nucleotides at each target nucleic acid sequence of said cell(s) via the guide(s). In some embodiments, mutation(s) include the introduction, deletion, or substitution of 40, 45, 50, 75, 100, 200, 300, 400 or 500 nucleotides at each target nucleic acid sequence of said cell(s) via the guide(s).

In some embodiments, for minimization of toxicity and off-target effect, concentration of Cas mRNA or protein and guide(s) delivered is controlled. In some embodiments, optimal concentrations of Cas mRNA or protein and guide(s) are determined, for example, by testing different concentrations in a cellular or eukaryote animal model and using deep sequencing the analyze the extent of modification at potential off-target genomic loci.

In some embodiments, technologies of the present disclosure provide a method of cleaving a target nucleic acid in a cell comprising contacting a cell with a Cas protein with collateral cleavage activity that is thermostable at temperatures above at least 60-65° C. and at least one guide sequence capable of hybridizing to at least one target nucleic acid, wherein the Cas protein is capable of forming a complex with the at least one guide sequence and causing a break in the at least one target nucleic acid.

In some embodiments, technologies of the present disclosure provide a method of altering expression of gene regulated or encoded by a target nucleic acid in a cell comprising contacting the cell with a Cas protein with collateral cleavage activity that is thermostable at temperatures above at least 60-65° C. and at least one guide sequence capable of hybridizing to at least one target nucleic acid, wherein the Cas protein is capable of forming a complex with the at least one guide sequence and causing a break in the at least one target nucleic acid.

In some embodiments, technologies of the present disclosure provide a method of altering expression of gene regulated or encoded by a target nucleic acid in a cell comprising contacting the cell with a Cas protein with collateral cleavage activity that is thermostable at temperatures above at least 60-65° C. and at least one guide sequence capable of hybridizing to at least one target nucleic acid, wherein the Cas protein is capable of forming a complex with the at least one guide sequence and editing the at least one target nucleic acid sequence.

In some embodiments, technologies of the present disclosure provide a method of modifying a target nucleic acid in a cell comprising contacting a cell with a Cas protein with collateral cleavage activity that is thermostable at temperatures above at least 60-65° C. and at least one guide sequence capable of hybridizing to at least one target nucleic acid, wherein the Cas protein is capable of forming a complex with the at least one guide sequence and editing the at least one target nucleic acid sequence.

In some embodiments, a method comprises binding of a CRISPR-Cas technologies to a target nucleic acid and effecting cleavage of a target nucleic acid. In some embodiments, CRISPR-Cas technologies cleaves target nucleic acid duplexes (e.g., DNA or RNA duplexes) by introducing double-stranded breaks. In some embodiments, a CRISPR-Cas technologies cleaves target nucleic acid duplexes (e.g., DNA or RNA duplexes) by introducing single-stranded breaks.

In some embodiments CRISPR-Cas technologies described herein comprise an exogenous donor template nucleic acid (e.g., a DNA molecule or a RNA molecule), which comprises a nucleic acid sequence of interest (e.g. a donor template nucleic acid sequence). In some embodiments, a donor template nucleic acid sequence is not identical to a genomic sequence that it replaces. In some embodiments, a donor template nucleic acid sequence may contain at least one or more single base changes, insertions, deletions, inversions or rearrangements with respect to the genomic sequence, so long as sufficient homology is present to support homology-directed repair. Without wishing to be bound by any one theory, upon resolution of a cleavage event induced with a CRISPR-Cas technologies described herein, the molecular machinery of the cell will utilize the exogenous donor template nucleic acid in repairing and/or resolving the cleavage event. Alternatively, the molecular machinery of the cell can utilize an endogenous donor template in repairing and/or resolving the cleavage event.

In some embodiments, a donor template nucleic acid sequence comprises sufficient homology to a genomic sequence at the cleavage site, e.g., 70%, 80%, 85%, 90%, 95%, or 100% homology, with the nucleotide sequences flanking the cleavage site, e.g., within about 50 base, 40 bases, 30 bases, 20 bases, 15 bases, 10 bases, 5 bases, or 1 base from the cleavage site. Without wishing to be bound by any one theory, homology with the nucleotide sequences flanking the cleavage site supports homology-directed repair between it and the genomic sequence to which it bears homology. For example, in some embodiments, approximately 25, 50, 100, or 200 nucleotides, or more than 200 nucleotides, of sequence homology between a donor and a genomic sequence (or any integral value between 10 and 200 nucleotides, or more) supports homology-directed repair.

In some embodiments, a useful donor template nucleic acid in accordance with the present disclose is not limited to a particular length; in some embodiments, a donor template nucleic acid is a nucleotide of any length (oligonucleotides or polynucleotides). For example, in some embodiments, a donor template nucleic acid comprises 10 nucleotides or more, 25 nucleotides or more, 50 nucleotides or more, 100 nucleotides or more, 250 nucleotides or more, 500 nucleotides or more, 1,000 nucleotides or more, 500 nucleotides or more, etc.

### Insertion

In some embodiments, CRISPR-Cas technologies as described herein are used to edit a target nucleic acid sequence by inserting one or more nucleotides into a target nucleic acid. In some embodiments, the insertion is a scarless insertion (e.g., the insertion of an intended nucleic acid sequence into a target nucleic acid results in no additional unintended nucleic acid sequence upon resolution of the cleavage event).

In some embodiments, insertions lead to frame shift mutations within a coding region of a target nucleic acid sequence that encodes a gene product. In some embodiments, CRISR-Cas technologies provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% insertion formation in the target nucleic acid.

In some embodiments, to calculate insertion frequencies, sequencing reads are scanned for exact matches to two 10-bp sequences that flank both sides of a window in which insertions can occur. For example, if no exact matches are located, the read is excluded from analysis. If the length of this insertion window exactly matches the reference sequence the read is classified as not containing an insertion. If the insertion window is two or more bases longer than the reference sequence, then the sequencing read is classified as an insertion. In some embodiments, the modifying entities provided herein can limit formation of insertions in a region of a nucleic acid. In some embodiments, the region is at a nucleotide targeted by a modifying entity or a region within 2, 3, 4, 5, 6, 7, 8, 9, or 10 nucleotides of a nucleotide targeted by a modifying entity.

In some embodiments, the number of insertions formed at a target nucleic acid can depend on the amount of time a nucleic acid (e.g., a target nucleic acid within the genome of a cell) is exposed to a modifying entity. In some embodiments, the number or proportion of insertions is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleic acid (e.g., a nucleic acid within the genome of a cell) to modifying entities. It should be appreciated that the characteristics of the modifying entities as described herein, in some embodiments, can be applied to any of the chimeric systems, or methods of using the chimeric systems provided herein.

### Deletion

In some embodiments, CRISPR-Cas technologies as described herein are used to edit a target nucleic acid sequence by deleting one or more nucleotides of a target nucleic acid.

In some embodiments, deletions lead to frame shift mutations within a coding region of a target nucleic acid sequence that encodes a gene product. In some embodiments, CRISPR-Cas technologies provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% deletion formation in the target nucleic acid.

In some embodiments, to calculate deletion frequencies, sequencing reads are scanned for exact matches to two 10-bp sequences that flank both sides of a window in which insertions can occur. For example, if no exact matches are located, the read is excluded from analysis. If the length of this deletion window exactly matches the reference sequence the read is classified as not containing an insertion. If the insertion window is two or more bases shorter than the reference sequence, then the sequencing read is classified as a deletion. In some embodiments, the modifying entities provided herein can limit formation of deletions in a region of a nucleic acid. In some embodiments, the region is at a nucleotide targeted by a modifying entity or a region within 2, 3, 4, 5, 6, 7, 8, 9, or 10 nucleotides of a nucleotide targeted by a modifying entity.

In some embodiments, the number of deletions formed at a target nucleic acid can depend on the amount of time a target nucleic acid (e.g., a target nucleic acid within the genome of a cell) is exposed to a modifying entity. In some embodiments, the number or proportion of deletions is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to modifying entities. It should be appreciated that the characteristics of the modifying entities as described herein, in some embodiments, can be applied to any of the chimeric systems, or methods of using the chimeric systems provided herein.

### Mutation

In some embodiments, CRISPR-Cas technologies as described herein can be used to edit a target nucleic acid sequence by mutating one or more nucleotides of a target nucleic acid. In some embodiments, a mutation is a point mutation. In some embodiments, a mutation is a silent mutation (e.g., the mutation does not lead to a change in amino acid sequence relative to a relevant reference amino acid sequence). In some embodiments, a mutation introduces a non-naturally occurring stop codon. In some embodiments, a mutation introduces a non-naturally occurring start codon. In some embodiments, a mutation removes a naturally occurring stop codon. In some embodiments, a mutation removes a naturally occurring start codon.

In some embodiments, it is desirable to generate and/or use chimeric systems (e.g., comprising a Cas protein and a modifying entity as described elsewhere herein) that efficiently modify (e.g., mutates or deaminates) a specific nucleotide within a target nucleic acid sequence, without generating a large number of insertions or deletions in the target nucleic acid sequence. In some embodiments, any chimeric system disclosed herein is capable of generating a greater proportion of intended modifications (e.g., point mutations or deaminations) than insertion/deletions.

In some embodiments, chimeric systems of the present disclosure modify a single nucleotide in a target nucleic acid. In some embodiments, the modification repairs and/or corrects a G-A or C-T point mutation, a T-C or A-G point mutation, or a pathogenic single nucleotide polymorphism.

In some embodiments, any of the chimeric systems disclosed herein are capable of efficiently generating an intended mutation, such as, for example, a point mutation, in a target nucleic acid sequence (e.g., a nucleic acid within a genome of a subject) without generating a significant number of unintended mutations, such as unintended point mutations. In some embodiments, any of the chimeric systems provided herein are capable of generating at least 0.01% of intended mutations (i.e. at least 0.01% base editing efficiency). In some embodiments, any of the chimeric systems provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, chimeric systems described herein are capable of generating a ratio of intended point mutations to insertion/deletions or unintended point mutations that is greater than 1:1. In some embodiments, the base editors provided herein are capable of generating a ratio of intended point mutations to insertion/deletions or unintended point mutations that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 10:1, at least 11:1, at least 12:1, at least 13:1, at least 14:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more.

In some embodiments, the number of intended mutations and insertion/deletions can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3: eaao4774 (2017); the entire contents of which are hereby incorporated by reference.

### Alteration in Gene Expression Level

In some embodiments, CRISPR-Cas technologies of the present disclosure are used to alter (e.g., increase or decrease) gene expression of gene products regulated or encoded by target nucleic acids. In some embodiments, gene expression levels are altered by, for example, gene or promoter insertion, deletion, mutation, gene expression inactivation, gene expression activation, enzyme engineering, directed evolution, knowledge-based design, random mutagenesis methods, gene shuffling, and/or codon optimization.

In some embodiments, alteration of gene expression levels includes targeting DNA. In some embodiments, targeting DNA comprises targeting regulatory elements. In some such embodiments, regulatory elements include, for example, promoters, enhancers, internal ribosomal entry sites (IRES), and other expression control elements (e.g., transcription termination signals, such as polyadenylation signals and poly-U sequences).

In some embodiments, alternation of gene expression levels includes targeting RNA. In some embodiments, targeting RNA comprises targeting RNA processing. In some such embodiments, targeting RNA processing includes targeting, for example, RNA splicing (including alternative splicing), RNA polymerase, viral replication, tRNA biosynthesis, and RNA activation.

### Payloads

In some embodiments, the disclosure provides methods of targeting insertion of a payload nucleic acid at a site of a target nucleic acid. In some such embodiments, the method comprises contacting a target nucleic acid with technologies described herein and a payload nucleic acid (e.g., a donor template nucleic acid comprising a payload nucleic acid). In some embodiments, the disclosure provides methods of targeting the excision of a payload nucleic acid from a site at a target nucleic acid, the method comprising contacting the target nucleic acid with technologies described herein.

In some embodiments, donor template nucleic acids are delivered either in a vector, such as an AAV viral vector, or as linear single stranded or double stranded DNA fragments. In some embodiments, for insertion of donor template nucleic acid by homology directed repair (HDR), donor template nucleic acids comprise a payload nucleic acid to be inserted into the locus of interest as well as flanking sequences that are homologous to endogenous sequences flanking the desired insertion site. In some embodiments, for insertion of short payloads less than for example, 1 kb in length, flanking homologous sequences can be short, for example, ranging from 15 to 200 nucleotides in length. In other instances, for the insertion of long payloads, for example, 1 kb or greater in length, long homologous flanking sequences are required to facilitate efficient HDR, for example, greater than 200 nucleotides in length. In some embodiments, cleavage of target genomic loci for HDR between sequences homologous to template DNA flanking regions can significantly increase the frequency of HDR. In some embodiments, cleavage events facilitating HDR include, but are not limited to dsDNA cleavage, double nicking, and single strand nicking activity.

In some embodiments, a payload nucleic acid is not limited to a particular nucleic acid; in some embodiments, a payload nucleic acid comprises any nucleic acid of interest. In some embodiments, for example, a payload nucleic acid is linear or circular. In some embodiments, for example, a payload nucleic acid is a plasmid, a viral genome, an RNA and/or DNA polynucleotide. In some embodiments, a payload nucleic acid is a modified nucleic acid. In some embodiments, donor template nucleic acids are double-stranded (e.g., DNA or RNA). In some embodiments, donor template nucleic acids are single-stranded (e.g., DNA or RNA). Methods of designing exogenous donor template nucleic acids are described, for example, in WO2016094874, the entire contents of which is expressly incorporated herein by reference. In some embodiments, a payload nucleic acid is a nucleic acid useful in treatment, prevention, and or diagnosis of a disorder and/or disease.

### Vector Systems and Vectors

In some embodiments, CRISPR-Cas technologies of the present disclosure include systems for delivering and/or expressing a Cas protein(s), chimeric system(s), guide(s), and/or donor template nucleic acid(s). In some embodiments, a system comprises a vector and/or vectors systems. Methods of delivering guides and donor template nucleic acids as well as methods for exogenously expressing proteins and polypeptides are well known in the art and the skilled artisan would recognize a variety of techniques could be successfully utilized.

Recombinant polynucleotides (e.g., DNA or RNA) encoding Cas proteins and/or chimeric systems or providing guides or donor template nucleic acids of the present disclosure may be prepared by a variety of methods available. For example, desired sequences may be excised from DNA using restriction enzymes, may be amplified from plasmids or genomic polynucleotide sequences using, for example, polymerase chain reaction, or may be synthesized using chemical synthesis techniques. In some embodiments, a combination of known methods is utilized to prepare a recombinant polynucleotide.

In some embodiments, recombinant polynucleotides encoding Cas proteins and/or chimeric systems of the present disclosure are cloned into a vector capable of expressing Cas proteins and/or chimeric systems. In some embodiments, recombinant polynucleotides providing guides or donor template nucleic acids of the present disclosure are cloned into a vector. Cloning may be carried out according to a variety of methods available (e.g., Gibson assembly, restriction digest and ligation, etc.). In some embodiments, a vector is a viral vector. In some embodiments, a vector is a non-viral vector. In some embodiments, a vector is a plasmid.

In some embodiments, a vector capable of expression comprises a recombinant polynucleotide that encodes a Cas protein and/or chimeric system of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of recombinant polynucleotides that encode a plurality proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of recombinant polypeptides are expressed, each of which is expressed from a separate vector.

In some embodiments, a vector comprising a recombinant polynucleotide encoding a Cas protein and/or chimeric system of the present disclosure is used to express a Cas protein and/or chimeric system by in vitro protein synthesis.

In some embodiments, a vector capable of expression comprising a recombinant polynucleotide encoding a Cas protein or chimeric system of the present disclosure is used to express a Cas protein or chimeric system in a host cell. In some embodiments, a vector capable of providing guide and/or donor template nucleic acids present disclosure is used to provide guides and/or donor template nucleic acids to a host cell. A host cell may be selected from a variety of the available and known host cells (e.g., Human Embryonic Kidney (HEK) cells, suspension HEK293 cells, Chinese Hamster Ovary cells) suitable expressing CRISPR-Cas technologies disclosed herein.

A variety of methods are known in the art to introduce a vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### Uses

CRISPR-Cas technologies as described herein have a wide variety of uses, including, for example, modifying (e.g., deleting, inserting, mutating, translocating, inactivating, or activating) a target nucleic acid sequence in a plurality of cell types and tissues and detecting target nucleic acids (e.g., DNA and/or RNA), for example, in specific high sensitivity enzymatic reporter unlocking (SHERLOCK)-based assays. In some embodiments, Thermostable Cas Proteins described herein are particularly useful in gene editing and/or detection of target nucleic acids in thermophilic organisms. Further applications include, but are not limited to, tracking and labeling of nucleic acids, enrichment assays (e.g., extracting desired sequence from a sample), detecting circulating tumor DNA, preparing next generation library, screening drugs, diagnosing and providing prognoses of diseases and disorders, treating various genetic diseases or disorders, and treating various non-genetic diseases or disorders, or augmenting health via manipulation of the genome.

### Gene Editing

In some embodiments, CRISPR-Cas technologies described herein are used for gene editing. In some embodiments, gene editing results in a gene silencing event, or an alteration of the expression (e.g., an increase or a decrease) in the expression of a desired target gene. Accordingly, in some embodiments, CRISPR-Cas technologies described herein are used in methods of altering expression levels of a gene product regulated or encoded by a target nucleic acid. In some embodiments, CRISPR-Cas technologies described herein are used in methods of modifying a target nucleic acid in a desired cell. In some embodiments, technologies disclosed herein provide methods for site-specific modification of a target nucleic acid in cells (e.g., eukaryotic or prokaryotic) to effectuate a desired modification in gene expression or function of the expressed gene product.

In some embodiments, the present disclosure provides engineered, non-naturally occurring CRISPR-Cas technologies comprising: a Cas protein with collateral cleavage activity that is thermostable at temperatures above at least 60-65° C.; and at least one guide sequence capable of forming a complex with the Thermostable Cas Protein and directing the complex to bind to at least one target nucleic acid.

In some embodiments, the present disclosure provides methods of cleaving at least one target nucleic acid in a cell comprising: contacting a cell with a Cas protein with collateral cleavage activity that is thermostable at temperatures above at least 60-65° C. and at least one guide sequence capable of hybridizing to the at least one target nucleic acid, wherein the Cas protein is capable of forming a complex with the at least one guide sequence and causing a break in the at least one target nucleic acid.

In some embodiments, the present disclosure provides methods of altering expression of at least one target nucleic acid in a cell comprising contacting the cell with a Cas protein with collateral cleavage activity that is thermostable at temperatures above at least 60-65° C. and at least one guide sequence capable of hybridizing to the at least one target nucleic acid, wherein the Cas protein is capable of forming a complex with the guide sequence and editing the at least one target nucleic acid sequence.

In some embodiments, the present disclosure provides methods of modifying at least one target nucleic acid in a cell comprising contacting a cell with a Cas protein with collateral cleavage activity that is thermostable at temperatures above at least 60-65° C. and at least one guide sequence capable of hybridizing to the at least one target nucleic acid, wherein the Cas protein is capable of forming a complex with the guide sequence and editing the at least one target nucleic acid sequence.

In some embodiments, the present disclosure provides methods of altering expression of at least one target nucleic acid in a cell comprising: contacting a cell with a Cas protein with collateral cleavage activity that is thermostable at temperatures above at least 60-65° C. and at least one guide capable of hybridizing to the at least one target nucleic acid, wherein the Cas protein is capable of forming a complex with the at least one guide and editing the at least one target nucleic acid sequence.

Accordingly, in some embodiments, the Cas protein has about 80%, 81%, 82%, 83%, 84%, 85%, 86%, 87%, 88%, 89%, 90%, 91%, 92%, 93%, 94%, 95%, 96%, 97%, 98%, 99% identity to any one of SEQ ID NOs: 1-10. In some embodiments, the Cas protein is identical to SEQ ID NO: 1. In some embodiments, the Cas protein is identical to SEQ ID NO: 2.

In some embodiments, methods of the present disclosure comprise contacting a CRISPR-Cas system of the present disclosure to at least one target nucleic acid and effecting cleavage of the at least one target nucleic acid. In some embodiments, CRISPR-Cas technologies cleave target DNA or RNA duplexes by introducing double-stranded breaks. In some embodiments, the CRISPR-Cas technologies cleaves target DNA or RNA by introducing single-stranded breaks or nicks.

In some embodiments, CRISPR-Cas technologies comprises a chimeric system with a modifying entity that modifies target DNA in a site-specific manner, where the modifying activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, the CRISPR-Cas technologies comprise a chimeric systems comprising a modifying entities or modifying entities that can edit DNA sequences by chemically modifying nucleotide bases, including deaminase enzymes that can modify adenosine or cytosine bases and function as site-specific modifying entities. Various modifying entities are known in the art and can be used in the method and systems described herein. Exemplary modifying entities throughout the present disclosure and are described in, for example, Rees and Liu Nature Review Genetics, 2018, 19 (12): 770-788, the contents of which are incorporated herein by reference.

In some embodiments, modifying entity activity results in the introduction of a stop codon or codons, for example, to silence a gene or genes. In some embodiments, modifying entity activity results in the removal of a stop codon or codons. In some embodiments, modifying entity activity results in the introduction of a start codon or codons. In some embodiments, modifying entity activity results in the removal of a start codon or codons, for example, to silence a gene or genes. In some embodiments, modifying entities result in altered protein function by altering amino acid sequences.

In some embodiments, Cas proteins of the present disclosure epigenetically modifying target nucleic acids by fusion with a histone. In some embodiments, Cas proteins epigenetically modify target nucleic acid by fusion with an epigenetic modifying enzyme such as a reader, writer or eraser protein. In some embodiments, Cas proteins are fused with a histone modifying enzyme to alter the histone modification pattern in a selected region of a target nucleic acid. Histone modifications can occur in many different ways including, for example, methylation, acetylation, ubiquitination, phosphorylation, and in many different combinations, leading to structural changes in DNA. In some embodiments, histone modification leads to transcriptional repression or activation.

In some embodiments, Cas proteins of the present disclosure modulate transcription of target nucleic acids by increasing or decreasing transcription through fusion with transcriptional activator proteins, transcriptional repressor proteins, small molecule/drug-responsive transcriptional regulators, or inducible transcription regulators. In some embodiments, CRISPR-Cas technologies are used to control the expression of a target coding mRNA (i.e. a protein encoding gene) where binding results in increased or decreased gene expression.

In some embodiments, CRISPR-Cas technologies are used to control gene regulation by editing genetic regulatory elements such as promoters or enhancers.

In some embodiments, CRISPR-Cas technologies are used to control the expression of a target non-coding RNA, including tRNA, rRNA, snoRNA, siRNA, miRNA, and long ncRNA.

In some embodiments, CRISPR-Cas technologies are used for targeted engineering of chromatin loop structures. Without wishing to be bound by any one theory, targeted engineering of chromatin loops between regulatory genomic regions provides a means to manipulate endogenous chromatin structures and enable the formation of new enhancer-promoter connections to overcome genetic deficiencies or inhibit aberrant enhancer-promoter connections.

In some embodiments, CRISPR-Cas technologies are used for live cell imaging. For example, in some embodiments, fluorescently labelled Cas proteins are targeted to repetitive genomic regions such as centromeres and telomeres to track native chromatin loci throughout the cell cycle and determine differential positioning of transcriptionally active and inactive regions in the 3D nuclear space.

### Methods of Treatment

As will be readily appreciated by those skilled in the art, in some embodiments, CRISPR-Cas technologies described herein are useful in one or more of various therapeutic applications. Accordingly, in some embodiments, a method of treating a disorder or a disease in a subject in need thereof is provided. In some such embodiments, the method comprises administering to the subject a Cas protein with collateral cleavage activity that is thermostable at temperatures above at least 60-65° C. and at least one guide sequence capable of hybridizing to a target nucleic acid.

In some embodiments, CRISPR-Cas technologies disclosed herein can be used to edit a target nucleic acid sequence to modify the target nucleic acid (e.g., by inserting, deleting, or mutating one or more nucleotides). For example, in some embodiments, CRISPR-Cas technologies described herein comprise an exogenous donor template nucleic acid (e.g., a DNA molecule or an RNA molecule) that comprises a desired nucleic acid sequence (e.g., a payload nucleic acid). Without wishing to be bound by any one theory, upon resolution of a cleavage event induced with CRISPR-Cas technologies described herein, the molecular machinery of the cell can utilize an exogenous donor template nucleic acid in repairing and/or resolving the cleavage event. Alternatively or additionally, in some embodiments, the molecular machinery of the cell can utilize an endogenous template in repairing and/or resolving the cleavage event. In some embodiments, CRISPR-Cas technologies described herein are used to modify a target nucleic acid resulting in an insertion, a deletion, and/or a point mutation. In some such embodiments, an insertion is a scarless insertion (i.e., the insertion of an intended nucleic acid sequence into a target nucleic acid resulting in no additional unintended nucleic acid sequence upon resolution of the cleavage event).

In some embodiments, CRISPR-Cas technologies disclosed herein are used to treat various diseases and disorders, e.g., genetic disorders, monogenetic diseases, disease that can be treated by nuclease activity, various cancers, etc. In some embodiments, methods described here are used to treat a subject, e.g., a mammal, such as a human patient. In some embodiments, the mammalian subject can also be a domesticated mammal, such as, and without limitation, a dog, cat, horse, monkey, rabbit, rat, mouse, cow, goat, or sheep.

In some embodiments, CRISPR-Cas technologies disclosed herein are used for correction of pathogenic mutations by insertion of beneficial clinical variants or suppressor mutations.

In some embodiments, CRISPR-Cas technologies disclosed herein are used for treating a disease caused by overexpression of RNAs, toxic RNAs, and/or mutated RNAs (e.g., splicing defects or truncations).

In some embodiments, CRISPR-Cas technologies disclosed herein target trans-acting mutations affecting RNA-dependent functions that cause various diseases.

In some embodiments, CRISPR-Cas technologies disclosed herein are used to target mutations disrupting the cis-acting splicing codes that can cause splicing defects and diseases.

In some embodiments, CRISPR-Cas technologies disclosed herein are used for antiviral activity, in particular against RNA viruses. In some embodiments, a RNA virus is a virus of, for example, the Arenaviridae, Arteriviridae, Astroviridae, Birnaviridae, Bornaviridae, Bunyaviridae, Caliciviridae, Coronaviridae, Flaviviridae, Filoviridae, Hepeviridae, Nodaviridae, Nymaviridae, Orthmyxoviridae, Paramyxoviridae, Picobirnaviridae, Picornaviridae, Pneumoviridae, Reoviridae, Rhabdoviridae, or Togaviridae family. In some embodiments, Cas proteins target viral RNAs using suitable RNA guides selected to target viral RNA sequences.

In some embodiments, CRISPR-Cas technologies disclosed herein are used to treat a cancer in a subject (e.g., a mammalian subject, e.g., a human subject). For example, Cas proteins described herein, in some embodiments, are programmed with a guide targeting an RNA molecule that is aberrant (e.g., comprises a point mutation or is alternatively-spliced) and found in cancer cells to induce cell death in the cancer cells (e.g., via apoptosis).

Further, in some embodiments, CRISPR-Cas technologies described herein are used to treat an infectious disease in a subject. For example, Cas proteins described herein, in some embodiments, are programmed with guide sequence targeting an RNA molecule expressed by an infectious agent (e.g., a bacteria, a virus, a parasite or a protozoan) in order to target and induce cell death in the infectious agent cell. In some embodiments, CRISPR-Cas technologies disclosed herein treat diseases where an intracellular infectious agent infects the cells of a host subject. In some embodiments, by programming the Cas protein to target a RNA molecule encoded by an infectious agent gene, cells infected with the infectious agent are targeted and cell death induced.

In some embodiments, CRISPR-Cas technologies disclosed herein are useful for the generation of cells for therapeutic delivery. In some embodiments, CRISPR-Cas technologies are useful for generating, for example, chimeric antigen receptor (CAR) T-cells, somatic cells (e.g., haematopoietic stem cells (HSC), mesenchymal stem cells (MSC)), and immortalized cell lines (e.g., neural stem cell line CTX). In some such embodiments, cells generated by CRISPR-Cas technologies are administered to a subject (e.g., for treatment of a disorder and/or disease).

In some embodiments, provided herein are compositions, pharmaceutical compositions, vectors, host cells, and kits comprising any of the proteins and/or polynucleotides of the engineered systems described herein.

### Collateral Activity Assays

Those skilled in the art will immediately appreciate that technologies provided herein are broadly applicable to achieve detection of a wide range of nucleic acids including, for example, nucleic acids from an infectious agent (e.g., a virus, microbe, parasite, etc.), nucleic acids indicative of a particular physiological state or condition (e.g., presence or state of a disease, disorder or condition such as, for example, cancer or an inflammatory or metabolic disease, disorder or condition, etc.), prenatal nucleic acids, etc.

In some embodiments, a target nucleic acid is detected by an assay comprising a Cas enzyme as described herein and a guide. In some embodiments, the structure of the guide can affect the activity of the Cas protein/guide complex. In some embodiments the structure of the Cas protein/guide complex contributes to the thermostability of the Cas collateral activity.

Those skilled in the art are well aware of the burgeoning plethora of useful detection (e.g., diagnostic) assays that have been and are being developed using Cas protein collateral activities. See, for example, Sashital Genome Med 2018:10, 32. Furthermore, those skilled in the art are well aware that a “detailed classification of CRISPR/Cas biosensing systems” based on Cas protein collateral activity has recently been made publicly available. See review by Li et al Trends Biotechnol. 37:730, July 2019.

Formats of particular interest include Cas13-based (e.g., Cas13a- or Cas13b-based) systems, including those referenced as “SHERLOCK” and/or “HUDSON” systems (see, for example, Gootenberg et al, Science 356:438, 2017; Gootenberg et al, Science 360:339, 2018; Myhrvold et al., Science 360:444, 2018; see also U.S. Pat. No. 10,266,887) and Cas12-based (e.g., Cas12a- or Cas12b-based) systems, including those references as “HOLMES” or “DETECTR” systems (see, for example, Cheng et al. CN patent filing CN107488710A; PCT/CN18/82769 and U.S. Ser. No. 16/631,157; Li et al. Cell Disc. 4:20, 2018; Chen et al. Science 360:436, 2018; Li, L. et al. bioRxiv Published online Jul. 26, 2018. http://dx. doi.org/10.1101/362889; U.S. Pat. No. 10,253,365). Both Cas13a and Cas13b enzymes have been used in SHERLOCK and/or HUDSON systems; similarly both Cas12a and Cas 12b.

As is known in the art, and described in references cited herein, typical detection assays that utilize Cas protein collateral cleavage activity involve contacting an appropriate CRISPR-Cas complex, including a Cas protein with collateral activity and a guide complementary to a target nucleic acid sequence of interest, with a sample that may contain the target nucleic acid. Upon recognition of the target nucleic acid sequence, the Cas protein's collateral activity is activated, so that it cleaves unrelated nucleic acid (DNA or RNA or both, depending on the enzyme). A reporter of the relevant cleavable nucleic acid is provided, appropriately configured (e.g., labeled) so that its cleavage as a result of the activated collateral activity is detectable (e.g., separates a fluorophore from a quencher so that fluorescence becomes detectable, etc.).

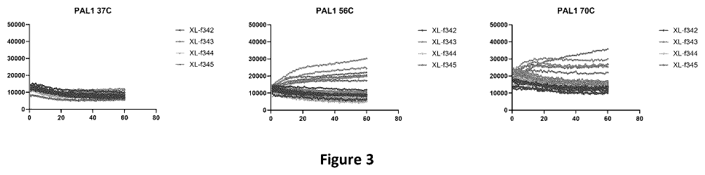

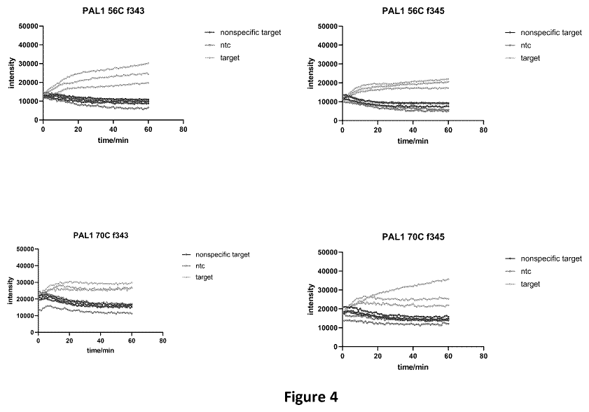

In many assays, a target nucleic acid sequence is generated and/or amplified (e.g., copied from RNA to DNA and/or amplified, for example by primer extension, DNA replication (e.g., by polymerase chain reaction) and/or transcription). See, for example, FIGS. 3 and 4 of the above-mentioned Li Review (Li et al Trends Biotechnol. 37:730, July 2019).

Thus, in many embodiments, a collateral activity assay includes steps of (1) target nucleic acid copying and/or amplification; (2) target nucleic acid binding; and (3) signal release and/or detection.

Typically, provided technologies will be applied to one or more samples to assess presence and/or level of one or more target nucleic acids in the sample. In some embodiments, the sample is a biological sample; in some embodiments, a sample is an environmental sample. In some embodiments, a sample is a crude sample (e.g., a primary sample or a sample that has undergone minimal processing).

In some embodiments, a sample will be processed (e.g., nucleic acids will be partially or substantially isolated or purified out of a primary sample); in some embodiments, only minimal processing will have been performed (i.e., the sample will be a crude sample).

Typically, collateral activity assays as described herein are in vitro assays. In some embodiments, they may be cell free assays (e.g., may be substantially free of intact cells, or, in some embodiments, of cell fragments).

In some embodiments, collateral activity assays as described herein are performed on samples that are or are prepared from biological (e.g., blood, saliva, tears, urine, etc.) or environmental (e.g., soil, water, etc.) primary samples.

In some embodiments, steps of nucleic acid detection and target binding are performed in a single vessel; in some embodiments, steps of target binding an signal release are performed in a single vessel; in some embodiments, steps of steps of (1) target copying and/or amplification; (2) target binding; and (3) signal release and/or detection are performed in a single vessel; in some embodiments all steps are performed in a single vessel—i.e., provided improved assays are one-pot assays.

In some embodiments, improved collateral activity assays as described herein are in vitro assays. In some embodiments, they may be cell free assays (e.g., may be substantially free of intact cells, or, in some embodiments, of cell fragments).

In some embodiments, improved collateral activity assays as described herein are performed on samples that are or are prepared from biological (e.g., blood, saliva, tears, urine, etc.) or environmental (e.g., soil, water, etc.) primary sample.

### Pharmaceutical Compositions

In some embodiments, the present disclosure, among other things, provides pharmaceutical compositions comprising CRISPR-Cas technologies of the present disclosure. In some embodiments, a pharmaceutical composition comprises a Cas protein with collateral cleavage activity that is thermostable at temperatures above at least 60-65° C. and at least one guide sequence capable of forming a complex with the Thermostable Cas Protein and directing the complex to bind to at least one target nucleic acid. In some embodiments, a pharmaceutical composition further comprises a donor template nucleic acid.

In some embodiments, a pharmaceutical composition comprises a vector or vector system that can express and/or provide CRISPR-Cas technologies of the present disclosure.

In some embodiments, CRISPR-Cas technologies of the present disclosure are formulated into pharmaceutical compositions by combination with appropriate pharmaceutically acceptable carriers or diluents.

In some embodiments, CRISPR-Cas technologies of the present disclosure are formulated into pharmaceutical compositions in a pharmaceutically acceptable vehicle. In some such embodiments, for example, pharmaceutically acceptable vehicles may be vehicles approved by a regulatory agency.

In some embodiments, vehicle refers to a diluent, adjuvant, excipient, or carrier with which a compound of the invention is formulated for administration to a subject. In some such embodiments, pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In some embodiments, auxiliary, stabilizing, thickening, lubricating and coloring agents are used. In some embodiments, pharmaceutical compositions are formulated into preparations in solid, semisolid, liquid or gaseous forms, such as tablets, capsules, powders, granules, ointments, solutions, suppositories, injections, inhalants, gels, microspheres, and aerosols.

In some embodiments, administration of CRISPR-Cas technologies described herein can be achieved in various ways. In some embodiments, administration comprises oral, buccal, rectal, parenteral, intraperitoneal, intradermal, transdermal, intratracheal, intraocular, etc., administration. In some embodiments, CRISPR-Cas technologies may be systemic after administration or may be localized by the use of regional administration, intramural administration, or use of an implant that acts to retain the active dose at the site of implantation. In some embodiments, CRISPR-Cas technologies are formulated for immediate activity or it may be formulated for sustained release.

In some embodiments, methods of treating comprise treating diseases or disorders of the central nervous system. In some such embodiments, it may be necessary to formulate CRISPR-Cas technologies in pharmaceutical compositions that cross the blood-brain barrier (BBB). For example, in some embodiments, drug delivery through the blood-brain barrier (BBB) entails disruption of the BBB, either by osmotic means such as mannitol or leukotrienes, or biochemically by the use of vasoactive substances such as bradykinin. In some embodiments, BBB opening to target CRISPR-Cas technologies to brain is used. In some embodiments, a BBB disrupting agent is co-administered with the therapeutic compositions of the invention when the compositions are administered by intravascular injection. In some embodiments, other strategies to cross the BBB are used, for example, use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein.

In some embodiments, CRISPR-Cas technologies are delivered behind the BBB by local delivery, for example, by intrathecal delivery.

In some embodiments, an effective amount of a preparation comprising CRISPR-Cas technologies is provided. In some embodiments, calculation of the effective amount or effective dose of a pharmaceutical composition as described herein to be administered is within the skill of one of ordinary skill in the art, and will be routine to those persons skilled in the art. In some such embodiments, the final amount to be administered will be dependent upon the route of administration and upon the nature of the disorder or condition that is to be treated.

In some embodiments, an effective amount given to a particular patient will depend on a variety of factors, several of which will differ from patient to patient. A competent clinician will be able to determine an effective amount of pharmaceutical composition to administer to a patient to halt or reverse the progression the disease condition as required. For example, in some embodiments, utilizing LD50 animal data, and other information available for the agent, a clinician can determine the maximum safe dose for an individual, depending on the route of administration. In some embodiments, for example, an intravenously administered dose may be more than an intrathecally administered dose, given the greater body of fluid into which the therapeutic composition is being administered. In some embodiments, pharmaceutical compositions are administered at higher doses, or in repeated doses, in order to maintain a therapeutic concentration. Utilizing ordinary skill, the competent clinician will be able to optimize the dosage of a particular pharmaceutical composition in the course of routine clinical trials.

In some embodiments, pharmaceutical compositions include, depending on the formulation desired, pharmaceutically-acceptable, non-toxic carriers of diluents, which are defined as vehicles commonly used to formulate pharmaceutical compositions for animal or human administration. In some embodiments, a diluent is selected so as not to affect the biological activity of the combination. In some such embodiments, for example, diluents are distilled water, buffered water, physiological saline, PBS, Ringer's solution, dextrose solution, and Hank's solution. In some embodiments, a pharmaceutical composition includes other carriers, adjuvants, or non-toxic, nontherapeutic, nonimmunogenic stabilizers, excipients and the like. In some embodiments, pharmaceutical compositions include additional substances to approximate physiological conditions, such as pH adjusting and buffering agents, toxicity adjusting agents, wetting agents and detergents.

In some embodiments, a pharmaceutical composition comprises any of a variety of stabilizing agents, such as an antioxidant for example. In some embodiments, wherein a pharmaceutical composition comprises a polypeptide, the polypeptide is complexed with various well-known compounds that enhance in vivo stability of the polypeptide, or otherwise enhance its pharmacological properties (e.g., increase the half-life of the polypeptide, reduce its toxicity, and enhance solubility or uptake relative to an appropriate reference standard). Without limitation, examples of such modifications or complexing agents include sulfate, gluconate, citrate and phosphate. In some embodiments, pharmaceutical compositions comprising nucleic acids or polypeptides of a composition can are complexed with molecules that enhance their in vivo attributes relative to an appropriate reference standard. Such molecules include, for example, carbohydrates, polyamines, amino acids, other peptides, ions (e.g., sodium, potassium, calcium, magnesium, manganese), and lipids.

In some embodiments, components used to formulate pharmaceutical compositions of high purity and are substantially free of potentially harmful contaminants (e.g., at least National Food (NF) grade, generally at least analytical grade, and more typically at least pharmaceutical grade). In some embodiments, compositions intended for in vivo use are sterile. In some embodiments, to the extent that a given pharmaceutical composition must be synthesized prior to use, the resulting product is typically substantially free of any potentially toxic agents, particularly any endotoxins, which may be present during the synthesis or purification process.

In some embodiments, pharmaceutical compositions are administered for prophylactic and/or therapeutic treatments. In some embodiments, toxicity and therapeutic efficacy of the pharmaceutical compositions is determined according to standard pharmaceutical procedures, such as in cell culture and/or experimental animals, including, for example, determining the LD50 (the dose lethal to 50% of the population) and/or the ED50 (the dose therapeutically effective in 50% of the population).

### Kits

In another aspect, the present disclosure provides kits containing any one or more of the elements disclosed in the above compositions and methods. In some embodiments, the kit comprises a vector and/or vector system as described herein. In some embodiments, the kit comprises one or more of the components of the CRISPR-Cas technologies as described herein, such as a Cas protein(s), guide(s), donor template nucleic acid(s) and/or a polynucleotide, vector, and/or vector system (e.g., DNA or RNA) encoding or providing the same. In some embodiments, the kit comprises instructions in one or more languages for using the kit. In some such embodiments, the instructions are to a specific application and/or method described herein. Elements may be provided individually or in combinations. Kits may be provided in any suitable container. In some embodiments, a suitable container is, for example, a vial, a bottle, or a tube.

In some embodiments, a kit comprises one or more reagents for use in a process utilizing one or more of the elements described herein. Reagents may be provided in any suitable container. For example, in some embodiments, a kit provides one or more reaction or storage buffers. Reagents may be provided in a form that is usable in a particular assay, or in a form that requires addition of one or more other components before use (e.g., in concentrate or lyophilized form). In some embodiments, a buffer is not limited to a particular buffer; in some embodiments, a buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In some embodiments, the kit comprises one or more of the vectors and/or one or more of the polynucleotides described herein. The kit may advantageously allow to provide all elements of the systems of the invention.

### EXEMPLIFICATION

### Example 1: Exemplary Thermostable Cas Protein Candidates

The present example describes certain Thermostable Cas Protein candidates.

TABLE 1
Exemplary sequences of Thermostable Cas Proteins.

| SEQ | | |
|---|---|---|
| ID | Cas | |
| NO: | Enzyme | Putative Sequence |
| 1 | Cas12a | MKSLAQFQNLYALQKTLRFELKPEGHTRETFNRWLEEIEKE |
| | | QASENENIVYQDLLRAKKYEKIKIILDEYHKDFIEQALAYAN |
| | | LTELEKYEELYRKSNRTSEEEEEFENTKESLRKQIANIFIKNP |
| | | NKTVQERWKFLFSKKLIQNELIVWVKGNYELLSEKLKNEFP |
| | | DESSIISTIEDFKYFTTYFRNYHENRKNLYSNEDKFSTIAHRLI |
| | | HENLPKFIDNIAIYQKAKAVLNINEVEKELGLPEDTLDKIFSL |
| | | DFFSKALTQKGIDQYNYFLGGKTENEVKKIKGLNEFINLYN |
| | | QQQQDKNQRLPFLKVLYKLPLFERTSTSFRFEPIENDRDLIE |
| | | RIGKFYYNDLKQYRDDSQGDTTDILSGINTLLRHVHDYRDG |
| | | LYVNGGITLTQISQKIFGSWSYINNALAYFYDTYIDASGVDH |
| | | QGERKPKKQKQIQEKTKWLKQKQFPVILVEKALSEYKSIET |
| | | NEDLKTRISDTTLCDFFKRCGNDDNGQDLFDRIEARLREKN |
| | | EEGYSLEDLLKKEFTTERKLMQDKTKTLLIKNFLDVIQGDK |
| | | DDITAGLLHFVKCLIPRTEISEKNELFYSGMEKYLNILSEVTP |
| | | LYNKARNYLTQKPYSIEKVKLNFENSTLLDGWDENEESDNS |
| | | CVLLRKRGYYYLGIMNKKHNMIFDRKIYPKATEGEAYYEK |
| | | MIYKLLPGAYKMLPKVFFSEKNIDYFKPSEEILRIRNTASYS |
| | | KNGQPQEGYQKASFSIEDCRKYIDFFKKCIANHWDWQKFN |
| | | FNFSPTEYYQSIDEFYREIERQGYKIDFVKIPESYINQLIKENK |
| | | LYLFKIYNKDFSEKKKSKGKDNLHTLYWKMLFDEKNLKDV |
| | | VLKLNGEAEVFFRQKSILYNEEIWNKGHHYSELKDRFSYPII |
| | | SNKRYAEDKFFLHVPITLNFKADGINNVNNMVNEFIKDNRD |
| | | IHIIGIDRGERHLLYVSVINQKGDIVEQCSLNEIVTEYNGKIFK |
| | | KNYHEELDNLEKERDRARKDWQTIANIKELKEGYLSHVIHK |
| | | ISKLILKYNAIVVMEDLNSGFKRGRQKVEKQVYQNFEKQLI |
| | | EKLNYLVLKESNVDEPGGVLRAYQLANKFETFKKLGKQSGI |
| | | IFYVPAAYTSAIDPVTGYIQYLYPLKQADSVEKARKFYSQFK |
| | | RISYNPHKQWFEFSFDYNDFNIIYHGKSSWTICTTNTERYM |
| | | WNRLLNNGHGGEELVYVTNELELLFGEYNIIYGDGKDIKQQ |
| | | ITDVQDIDVDRTAKQFYKRINELLNLTLKLRHNNGKKGADE |
| | | EDYILSPVEPYFDSRFESRKPSMQQTLPINADANGAFNIARK |
| | | GLLLLERLNQLGVEEFEKTKKSNNKKTQWLPHELWVEYAQ |
| | | NHTRK |
| 2 | Cas12a | MQDKTGWSSFTNKYSLSKTLRFELKPVGNTQKMLEDDGVF |
| | | QKDRERQENYKKVKPFMDKLHREFIKEALNNLKLEGLTEY |
| | | FEIFKKFRKDKNNKELKNAEKKLRQIIGRCYTETAQIWVEK |
| | | YKEFGFKKKNIGFLFEEGVFELMKLKYGNDEASQIEKNGEV |
| | | LSIFDGWKGFLGYFKKFFETRNNFYKDDGTSTAVSTRIINEN |
| | | LKIYLDNLIKYNKIKDKVDFKEADILQENKLNLSDFFNVESY |
| | | AKYSLQKGIDYYNEILGGKTLKNGTKLKGLNEVINEYKQK |
| | | NKSGELSKFKMLKKQILGEGEDRTLFEEIENEDELKDVLKD |
| | | FFYNADPKITLFKTLLEDFFSNTEKYKDELDKIYFNTVAINGI |
| | | LHRWVDDSGVFQKYLFEVLKSNKLVKSNHYDKKEDSYKFP |
| | | DFISFEHIKVALENCERDGLKDKFWKEKYYTKECLTENGLA |
| | | NLWQEFLEIYKCEFKKLYDYKTDDNDCYLQYRDNYKKYIL |
| | | DANFNPKEKSAKDIIKDYLDSVLSIYQLAKYFALEKKKVWT |
| | | TDYETGDFYYEYIKFYEDTYEQIIKPYNLVRNYLTRKPINTA |
| | | KKWKLNFDNAYLASGWDKDKEVSNLTVILRRDEQYYLAI |
| | | MKKGKNKIFEKKFSCGEFEKMEYKQIAEASSDIHNLVLMND |
| | | GSCRRCIKMHDKRKYWPLDISIIKEKKSYAKENFVRRDFERF |
| | | VNYMKKCSLLYWKEYDLKFSDTSTYKNINDFTNEIASQGY |
| | | KLSFSAIPESYINEKNNNGELYLFQIYNKDFGIKTEGNKNLH |
| | | TMYWESIFSEENRFRNFIVKLNGKAEIFYRPKSEQVEKEQRN |
| | | FTREIIKNRRYTENKIYFHCPITLNRISRENVKKENNGINNYIA |
| | | TNPNINILGVDRGEKHLVYYAIVDQDGKLIDAEDATGSFNTI |
| | | GSTDYHRLLEEKAKDREKERKDWDLIRGIKDLKKGYISLVV |
| | | RKIADLAIKYNAIIIFEDLNTRFKQIRGGMEKSVYQQLEKALI |
| | | NKLSFLVNKGEKDPEQAGHLLKAYQLAAPFQTFDKMGRQT |
| | | GIIFYTQASYTSKIDPITGWRPNLYLKYRNIDDSKESIKKFKSI |
| | | LFNKEKNRFEFTYDLKDFVDFEEDKIPEKTEWTLCSSVERH |
| | | KWNRHMNNNKGGYEVYKDLTENFYKLFDENNISMNKDIV |
| | | DQVESISNGNFFRQFIYLFNLVCQIRNTDEKAEDVDKRDFIL |
| | | SPVEPFFDSRRAKDFKAYGDNLPKNGDENGAYNIARKGVLI |
| | | IKKIKEYYNQNGSCDKLGWGDLSISHKEWDDFATNN |
| 3 | Cas12a | MDSYEQFTKLYPIQKTIRFELKPQGRTKEHFDNSNFLEKDRE |
| | | RDDNYKILKEVIDDYHREFIDECLSNIQLNWDDLKKFSEEYR |
| | | RSKEKKNNRDSESEQKRMSTTSETRAINKKNLEAEQKRMR |
| | | GEIVSAFKKDDRFKHLFSEKLFSILLKNQIYEKGTLEEIEAFD |
| | | CFNKFSGYFKSFHENRKNMYSDEDKETAISYRIINENFPKLL |
| | | DNFEKYQYVCREYPEQIREAESTLAEAGCYIKMDEIFSIDNF |
| | | NNVMMQGGKESGISRYNLAIGGIVQGTGEKPKGLNEFLNL |
| | | AYQNEPNGRKKIRMEPLYKQILSKEESFSYRLEAFTDDSQLL |
| | | SAIRSFFDIVEKDKNGNIFDRAVNLMSSFSNYDTSKIYIRKAY |
| | | LNQVSKEIFGYRGKSDSKPAKTADESLNKSGGWEKLGQML |
| | | RDYKADSIGDRNLEKTCKKVDKWLDSDEFTLSDILGAISLA |
| | | GSNETFEAYVSEICVARRNIDKEKEKEKNINVEKISGDTESIQ |
| | | IIKALLDSVQEFFHLLSPFQLHPNTPHDWTFYAEFNDIYDKL |
| | | SAITPLYNQARNHLTKKNLDTSKIKLNFNNPTLANGWDVN |
| | | KEYENTAVILIRDGKYYLGIMNPKNKRKIKFDEGSGAGPFY |
| | | QKMVYKLLPGPYRMLPKVFFAKKNIDYYNPSQEIREGYKA |
| | | GKHKKGKEFDKGFCHKLIDFFKESIQKNENWKVFDFKFSPT |
| | | ESYDDISEFYQEVEKQGYRMYFVNIPSDTIDRYVEGGDMFL |
| | | FQIYNKDFAKGAKGNKDMHTLYWNAVFSEENLQKGVMKL |
| | | SGEAELFYRKKSDIKDPPHREGEILVNRTYIDRTHVSGVMGE |
| | | QNTVKESRIPVPDEIHKNLFDYYNHGRELTKEEKEYCDKVG |
| | | SFKAYYGIVKDRRYLENKMYFHVPLTLNFKAIGEKRINKMA |
| | | IEKFLTDENACIIGIDRGERNLLYYSIIDRNGKIIDQKSLNVID |
| | | GFDYHEKLSQRQTEREVARQSWNSIGKIKDLKEGYLAKAV |
| | | HEISKMAIKYNAIVVLEDLHFGFKKGRLKVEKQIYQKFEEM |
| | | LINKLNYLVFKDVSDSSDAGGVLNAYQLTAPLESFSKLGKQ |
| | | SGILFYVPAAFTSVIDPTTGFVDLFNSSSITSTQKKKEFLQRFE |
| | | SIVYSARDGGIFAFTFDYRNFSKIATDHRNMWTVYTHGERI |
| | | RYVRDEKCYKTTDPTKRIKEALSGIEYDDGSDIRDKITQSGD |
| | | NNLINTVYHSFMDTIKMRNKDGRIDYIISPVKNRNGEFFRSD |
| | | YKHRDFPVDADANGAYHIALKGELLMRMIGKTYDSNSDK |
| | | MPKLEHKDWFEFMQTRGDQ |
| 4 | Cas12b | MCVSRLPWFNITLTGKLNRQRLNQMCVSRLPWFCTPKGQL |
| | | AATPKTVVAQQENAMLAIIRDVHEAAPADLKTVAQRLEPG |
| | | YFVTQFPKQQMTGDEARAEAERLFAACQKKFKELAEYEDG |
| | | YRQCLDALGPNLSLPRLGRKPKGAYPYAVVFKLMPTNATW |
| | | ECFKRVTASLYKRAQKGVVSPVSADSIADVRINDEPLFEYF |
| | | TNLALVRPPGNKDRAVWFEFDLAAFIEAIKSPHQFFQDTIKR |
| | | EQAVAQIKAKLDAMDGQGRAASGEEDALPGFEGDDRITLL |
| | | RELVTDTLGYLAEADASTSPGGKIEYSIQERTVRGFAEVKRR |
| | | WRDLVEKGKATEDALLKVLAEEQTEHRDDFGSATLYRELA |
| | | KPKFQPIWRDPGTQPWHADDPLRAWLEYRELGRELEDKQR |
| | | PIRFTPVHPVHSPRFFIFPKKKGGGRFGTVHEPGQLRVMAGI |
| | | VAQTQHGWEPVPVRITYAAPRLRRDQLRDDVETDLESRPW |
| | | LQPMMQALGLPEPDTADFSNCRVTLQPSAPDDIQLTFPVDV |
| | | SADKLTTAIGKAARWAKQFNLFPDGDNFYNASLRWPHEKK |
| | | PSKPPVPWHEALDNFSVLAADLGQRCAGAFARLEVRANDD |
| | | FAGKPSRFIGETPGKKWRAALVAAGMLRLPGEEQTVWRPG |
| | | ATGPNFHTELSGSRGRMARPHEADDTADLLRAFDCPEESLM |
| | | PADWRTSLSFPEQNDKLLVAARRYQSRLARLHRWCWFLTD |
| | | EKKRQTALDEIREAEDMPAADDPQLTDKLRALLLQKQAAL |
| | | PGLLVRLANRILPLRGRSWQWETHPDKADCHLLTQTGPALP |
| | | DVWIRGQRGLSMQRIEQIEELRRRFQSLNQMQRREIGGKPPI |
| | | RRDDSIPDCCPDLLDKLDQIKEQRANQAAHMILAEALGLRL |
| | | APPPADKRQLRASRDVHGQYVKSREPVDFIVIEDLSRYRSSQ |
| | | GRAPRENSRLMKWCHRAVRDKLRELCEPFGIPVVETPAAYS |
| | | SRFCSRSGVAGFRAVEVGPGFDREFPWMMLKDREDEGEPV |
| | | RQLILQVATLNQGRDGKPPRTLLAPLAGGPIFVPIVDKLNGA |
| | | DIQPALAQADINAAINLGLRAIADPRLWSIHPRCRTQRQGDQ |
| | | MLTREKRKFGETGQPLAVHRADGVKPDDTRNPNFFADISGS |
| | | LPAWESATLDGQHLLSGRCLRSEIKKRQWQRCAEINDRRM |
| | | NRWMKGE |
| 5 | Cas12b | MTELQTQRAYTLRLKGIDEKDQSWRDALWKTHEAVNKGA |
| | | KVFGDWLLTLRGGLDHTLADAEIPGEKGKPDRAPTQEERK |
| | | HRRILLALSWLSVESERGAPEEFIVATGKEPAATRNDKVIAA |
| | | LKDILRGRNLTEEKISEWTEVCTPSLSAAIREDAVWVNRSRA |
| | | FDEAVKRIGSSLTREEVWDMLECFFGSRNAYLAPVKISEDES |
| | | SDGEQEEKAKDLVQKAGQWLSSRFGTGEGADFAKMAAVY |
| | | AKIAAWAGNAQAGTTGNEVINNLATALREFTPKSNDLKGV |
| | | LDLISGPGYKSATRNLLKQIANTKTVTREDISKLQETAGEDS |
| | | EECATKTGSKGKRAYADAILKDVESVCGFTYRIDKDGQPVS |
| | | VADYSKYDEDYKWGSSRHKEFAVMLDHAARRVSLAHTWI |
| | | KRAEAERRKFEEDSKKIMQVPQAAKDWLDAYCAQRSEASG |
| | | ALEPYRIRKRAIQGWKEIIASWNKPDCKTAEDRIAAARQLQ |
| | | DDPEIEKFGDIQLFEALAEDDAQCVWKKEDGTLDPEILINYT |
| | | LASEAMFKKQHFKVPSYRHPDAFLYPVFCDFGNSRWELDFS |
| | | IREAATKLKEIEAKIEKQRQEVHKVQQALEKCENDEKRPKM |
| | | EERLKEAQKKLQESQNYGEYLHSNNKITMVLFDGTFVKKHI |
| | | FAWQSKRLTKDLALYQEPSADPKNVVSRADRLGRAVASVG |
| | | INDAVKVAGLFEQENWNGRLQAPRQQLEAIAQYVEKHGW |
| | | DNKAEKMRASIKWFITFSAKLQSKGPWNEFARKHGLKEDP |
| | | HYWPHAEKNENRTAHSRLILSRLPGLRVLSVDLGHRYAAA |
| | | CAVWEALGSEAFKKDIEGKRIIRGDTDENALYCHTEHEANG |
| | | KKHITIYRRIGADTLPDGAHHPAPWARLDRQFLIKLQGEDE |
| | | QAREASNEEIWKVHQLENTLGRRTPLIDRLIAGGWGYTEKQ |
| | | KARLEVLTNLGWCPTNKTDNQEEGDEEETAILSKPSLLVDD |
| | | LMFSAVRTLRLALKRHGDRARIAHYLITDEKTKPGGVKEKL |
| | | DKNGRVELLLDALGLWHDLFSSPGWHDEKAKQLWNAYIA |
| | | GLLPEGELQQAKSVTTSAALGGQQKKEKKEKLRAVAEALY |
| | | LNSDLCHSLNEVWRKRWEEDDKQWRIYIRWFKDWIMPRG |
| | | ANAKSPAIRHVGGLSLTRLATLTEFRRKVQVGFFTRLHPDG |
| | | TKTETREDFGQKTLDTLEHLREQRVKQLASRIVEAALGIGSE |
| | | DKRHWDGKKRPRQRIADPRFVPCHAVVIENLTHYRPEETRT |
| | | RRENRQIMEWASSKVKKYLSEICQLHGLHLREVSAAYTSHQ |
| | | DSRTGAPGIRCQDVSLIEFMKSPFWRKQVAQAEKKQKEGK |
| | | GDAVERYLCELNQKWKGASEEEWRKAGFVRIPLRGGEIFV |
| | | SAAGHDSPAAKGIHADLNAAANIGLRALLDPDWSGKWWY |
| | | VPCNSSTMCPARDKVTGSAAVNPGQPLQVSAQLESDDAAK |
| | | DTKKRKKKGDGKSKEIINLWRDISSYPLEDTRGGTWSNKTV |
| | | YWNRVQSNVVHILQNQMKG |
| 6 | Cas12b | MPETTQRAYTLRLQGHDPKDASWREALWKTHEAVNRGAK |
| | | AFGDWLLTLRGGLDHSLADEGAPGQTPTEEQRKQRRILLAL |
| | | SWLSVESENGAPQEYIVPHDRDNESGARQNWKTREALREIL |
| | | KNRGCRDDEIESWCHDCEPSLTSAIRKDAVWVNRSKAFDN |
| | | AVQSIPNFSREEIWDLLGCFFVSSQAYLAPLESPKDDKPDAS |
| | | KKDSSKDLIQSAGQWLSRRFGRGKGLNFARLAETYEAIAR |
| | | WASVANPGDTNDLIADLAKTLNAETPELDGILKVVSGPGHK |
| | | SKTRNLLRSLSAVNHITKDTLQRLKDTANEDAKKAKLKKG |
| | | EKGHRAYAYKVLEAVEDACGFTYLQEGDRAKHCEFAVML |
| | | DHAARRVSSLHTWIKRAEAERRRFEIDTKKKDQLPPSVKEW |
| | | LDTYCQKRSKETGAVEPYRIRRGAIEGWKEIVEAWSKAGTT |
| | | TAEDRKHEARRLPDNPHIDKSGDIKLFEDLALEDALPVWHA |
| | | NGDPNNPPDPQLLIDYVEGSEAEFKKRAFKVPTYCHPDPLV |
| | | HPVFCDYGCSRWNVSFAIQPVKKQKLSSEEKLPAKGLLLDL |
| | | LHGTAIRPVALRWQSKRFARDLALNTTDSSDKPNEVTRADR |
| | | FGCALAKCPSSQKIRIRGLFEEKYWNGRLQAPRPELTALAK |
| | | RVAKYGWDKKARKLRNSLNWFITFSANLRPSGPWEEYTKY |
| | | AEKAFSSNASAKPSVSRGGFWVVHASPNKRGKMAQLRLCR |
| | | LPELRVLSVDLGHRYAAACAVWETLSKSAFEQEIHERKILR |
| | | GGTGPNDLFCHTQHDTNGQSKVTIYRRIGADTLPNGTPHPA |
| | | PWARLDRQFLIKLPGEEREARKASPTELANVEKLEKELGLK |
| | | TSENRVKRIDDLMSDTLRTVRQALRRHSLRARIAFNLATLR |
| | | DQSDGDEESQSKQKRDTRWNNTVKIWHSLLESNEWTDDW |
| | | AKALWDELGPLSDPQKADDAEWLKLAAEKFYTRWQEDEQ |
| | | TWRERLRWLRRWILPRGSQAASQKGSIRHVGGLSLTRLATI |
| | | KTLYQVLKAYHMRLKPDNSRKNIPAEGDEALQNFGQKILD |
| | | DLEHMREQRVKQLASRIVEAALGLGRMKQVTIGKDPKRPR |
| | | EPVDQSCHAVVIENLTHYRPEKRQTRRENRQLMDWSAAKV |
| | | KKYLKECCQLHGLHLVEVSASYTSRQDSRTGAPGIRCQEVP |
| | | LTDFLKKNFWREQVKQAKQRLSEGKANARDRYLCQLNER |
| | | WGNAPAPVTQTAIRLRIPLNGGELFVSADQNSPASKGIQAD |
| | | LNAAANIGLRAITDPDWPGAWWYVPCEANTFKPVKDKVA |
| | | GSAAIDSNVSLKKDSPNSEKPASDRKSRTSKSMINLWCDTSS |
| | | KSLSEKDQWQESAPYWEDVAARTINILQASLACSTTNSQ |
| 7 | Cas12a | MKSLAQFQNLYALQKTLRFELKPEGHTRETFNRWLEEIEKE |
| | | QASENENIVYQDLLRAKKYEKIKIILDEYHKDFIEQALAYAN |
| | | LTELEKYEELYRKSNRTSEEEEEFENTKESLRKQIANIFIKNP |
| | | NKTVQERWKFLFSKKLIQNELIVWVKGNYELLSEKLKNEFP |
| | | DESSIISTIEDFKYFTTYFRNYHENRKNLYSNEDKFSTIAHRLI |
| | | HENLPKFIDNIAIYQKAKAVLNINEVEKELGLPEDTLDKIFSL |
| | | DFFSKALTQKGIDQYNYFLGGKTENEVKKIKGLNEFINLYN |
| | | QQQQDKNQRLPFLKVLYKLPLFERTSTSFRFEPIENDRDLIE |
| | | RIGKFYYNDLKQYRDDSQGDTTDILSGINTLLRHVHDYRDG |
| | | LYVNGGITLTQISQKIFGSWSYINNALAYFYDTYIDASGVDH |
| | | QGERKPKKQKQIQEKTKWLKQKQFPVILVEKALSEYKSIET |
| | | NEDLKTRISDTTLCDFFKRCGNDDNGQDLFDRIEARLREKN |
| | | EEGYSLEDLLKKEFTTERKLMQDKTKTLLIKNFLDVIQGDK |
| | | DDITAGLLHFVKCLIPRTEISEKNELFYSGMEKYLNILSEVTP |
| | | LYNKARNYLTQKPYSIEKVKLNFENSTLLDGWDENEESDNS |
| | | CVLLRKRGYYYLGIMNKKHNMIFDRKIYPKATEGEAYYEK |
| | | MIYKLLPGAYKMLPKVFFSEKNIDYFKPSEEILRIRNTASYS |
| | | KNGQPQEGYQKASFSIEDCRKYIDFFKKCIANHWDWQKFN |
| | | FNFSPTEYYQSIDEFYREIERQGYKIDFVKIPESYINQLIKENK |
| | | LYLFKIYNKDFSEKKKSKGKDNLHTLYWKMLFDEKNLKDV |
| | | VLKLNGEAEVFFRQKSILYNEEIWNKGHHYSELKDRFSYPII |
| | | SNKRYAEDKFFLHVPITLNFKADGINNVNNMVNEFIKDNRD |
| | | IHIIGIDRGERHLLYVSVINQKGDIVEQCSLNEIVTEYNGKIFK |
| | | KNYHEELDNLEKERDRARKDWQTIANIKELKEGYLSHVIHK |
| | | ISKLILKYNAIVVMEDLNSGFKRGRQKVEKQVYQNFEKQLI |
| | | EKLNYLVLKESNVDEPGGVLRAYQLANKFETFKKLGKQSGI |
| | | IFYVPAAYTSAIDPVTGYIQYLYPLKQADSVEKARKFYSQFK |
| | | RISYNPHKQWFEFSFDYNDFNIIYHGKSSWTICTTNTERYM |
| | | WNRLLNNGHGGEELVYVTNELELLFGEYNIIYGDGKDIKQQ |
| | | ITDVQDIDVDRTAKQFYKRINELLNLTLKLRHNNGKKGADE |
| | | EDYILSPVEPYFDSRFESRKPSMQQTLPINADANGAFNIARK |
| | | GLLLLERLNQLGVEEFEKTKKSNNKKTQWLPHELWVEYAQ |
| | | NHTRK |
| 8 | Cas12b | MAYQNGKEQPTVTNQRAYTLRLSGTNDQDSIWRNRLWHT |
| | | HEAVNKGAKTFGDWLLTMRGGLCHTLAEADVPGKGNKPA |
| | | RHPTPQEIRSRRVVLALSWLSVESQHGAPERHLVSHDLDIAT |
| | | GERKNWKTVEALREILHGRCLCKELIDEWANDCRDSLSATI |
| | | REDAVWVNRSKAFDLAAKKIGASLTREELWDFLQPFFANK |
| | | HGYLQMDTVAGVTNGDSETDAEEAKEDSSEEKAKDLSQK |
| | | AGQWLSSRFGTGTGADFSRFSKVYEVLAARCGSVAVGVSG |
| | | VEAIRILAGTLADFSPGSNDIEGMLGLMSGPGYKSATRNILQ |
| | | KINTLQTVSQQDLDRLREASEKDALQSKQKVGGKGSRPYA |
| | | NAILQDVEAACGICYAGTGESPARHWQYAVILDHAARRVS |
| | | MAHSWIKRAEEQRSKFEIEKDKLDHVPKDALAWLDAFCAR |
| | | RSSESGASDAYRIRRSAVDGWKQVVAAWAALPPKPENQGS |
| | | ELLSDAESARIQAARELQDTVEKFGDIQLFEALSLTGAKCV |
| | | WQPDGRPDAQPLLDYVAGTDAISKKQRFKVPAYRHPDALL |
| | | HPVFCDFGNSRWNINYAIHRAPEKLTPAQQLLEKKKAEIDK |
| | | AELTLAKAGDAAKQANISEKINGLRAAFIQQQEKVAWLNS |
| | | RHAMTMSLWDGTHIEDTPLIWQSKRFGSDIGQPVEAQPLPV |
| | | SRADRFGRAVALAQDNVPVIPSGLFDLSDWNGRLQAPRRQ |
| | | LEAIAAIRDSAKLSVNEKQQLVAKRIQSIRWLLTFSAKLQSH |
| | | GPFIAYAAQHGFDWRYGAHGPENKSRQGLAKLILCRLPGLR |
| | | ILSVDLGHRYAAACAVWETLNAGQIQKACLDAGKEAPGPC |
| | | TLYLHLKQIANGKEKKTIFRRIAADTLPDGSPHPAPWARLD |
| | | RQFLIKLQGEDRDARLATSEEIAAVEQMENELGVVRQLKRK |
| | | GRELLVDELMSDALRTLRLGLRRHGVRARIAFNLTANKRIR |
| | | PGGKEEVLDQEGRVLLLTETLLAWYELYTAERWTDEPARE |
| | | LWNRHIQPLLGATILQNTVNQEDTPSAAKRRKLREETSGKL |
| | | KHVAEEIAKNDSLCRQLHVLWSAQWQTEDVIWRTRLRMM |
| | | RRWLLPRGVKRNAQLRISIRDVGGLSLTRIASFKSLYQVQK |
| | | AYQMRPHPEDPRLNIPERGDSRLENFGQRVLDAMERMREN |
| | | RVKQLASRIAEAALGIGGETGISSKDGSQKKRPTERSSDPRF |
| | | APCHAVVIEDLTHYRPDETQTRRENRQLMSWSSSKVKKYL |
| | | GEACELNGLYLREVSPAYTSRQDSRTGAPGLRCNDVTVVEF |
| | | NNSPFWRKQVGAAEKNQKEGNKGDARERYLLSIEEGIRGA |
| | | ANDRDIFRIPVKGGEIFVSACITDGGNNAKKNAPPGLQADL |
| | | NAAANIGLRAIFDPDWEGRWWYIPCDAATLCPDAKKFIGC |
| | | KAVDPTKPLRVVAEEGAISASGIGSKKSGRKKNAATDGTRI |
| | | VNLWRDPSGAPIHRDVLRSPEWQDYAGYWNEVQHRVIRNL |
| | | KTCYEQTSQQEDPFVSQDADKPF |
| 9 | Cas12b | MKRLAETALADKVKCETNSRPKGERAYANSILHDVESACG |
| | | FTYRVDKGEQPVPVSDYSHYANDYRWGPANHSEFAVMLD |
| | | HAARRVSLAHTWIKRAEAERRQFEENAKKIDKVPKVAREW |
| | | LDSLCAERSIVLGALEPYRIRRRAVDGWKHVVAAWSKSDC |
| | | KTAQDRITAARLLQEDPEIDKFGDIQLFEALAEDHAVCVWQ |
| | | RDGEAGKTSDPQLLIDYALAAEAEFKKRHFKVPAYRHPEAF |
| | | WHPVFCDFGQSRWKICFDVHKNRQSRRQRACANRISRKICF |
| | | DVHKKRQTLRLSLEVWTGSKMLDMPLCWQCKRLARDLAL |
| | | GQDHKKDRSCQVTRADRLGRAVSNVARNQEVQILGLFEQE |
| | | YWNGRLQAPRPQLEALGRYIEKHGWDAKAQKSCRAIRWM |
| | | ISFSPRLQPAGPWGKFAEKLQLNPNPKYWPHAEDNKDRGSR |
| | | SKLILCRLPGLRVLSVDLGHRYAAACAVWEAVDAEQVKEA |
| | | CQAAGHREPNENDLYLHLKKRTTKQKKGSQGVVEETTIYR |
| | | RIGADTLPDCTPHPAPWARLDRQFLIRLQGEEDEARAASNE |
| | | EVWAVHKLEAELGRTIPLIDRLLGAGWGQTEKQKARLKAL |
| | | RELGWTPANKCQAFNSTDETELRRPSLAVDELMLDAVGTL |
| | | RLALKRHGDRARIARYLITDERTKPGGVKEKLDENGRIELL |
| | | QDALIIWHGLFSSPRWRDDAAKQLWNEHIAKLVGEQNLVE |
| | | VSEDASGSERRTKQKQNREKLREAAKALVDDVALRQALHD |
| | | MWKRRWEEEDREWRRRLRWFKDWVLPRREQARKAYSRP |
| | | AETGSSSHPKRRARYAAIRRVGGLSLTRLATLTEFRRKVQV |
| | | GFFTRLKPDGTKAEAKEGFGQSTLDALEHLRAQRVKQLAS |
| | | RIVEAALGVGRIRRFPGVKNPKRPDTPVDKPCHAIVIENLTH |
| | | YRPEETRTRRENRQLMTWSSSKIKKYLAEACQLYGLHLREV |
| | | TAAYTSRQDSRTGAPGLRCQDVPVKEFMRSLFWRKEVAQA |
| | | EKKLTAGKGSSYERLLCELNQRWKDNSPGDGKRAELLRLP |
| | | HKGGEIFVSAAPDSPAARGLQADLNAAANIGLRALTDPDWP |
| | | GKWWHVPCNAVTFRPVEDKVKGSAAVKLDQSLRQVAHPQ |
| | | SKDPGAKKSKEIVNLWCDISSLPLEHREWKLDWEPYPAYW |
| | | NNVQCRVIRVLQGKV |
| 10 | Cas12b | MANAKVKTTTRSYTLSLNAPSDTTDRSPLWHRIFRTHYAIC |
| | | CGAREFGKLLLDLRGGLPTSLAQLGEGIAENDRRQTQRGTR |
| | | RILALGWLSVEDLDHARNDPHRVQDTAPGSPLDQDLAEKIL |
| | | RKILITKGIKSEEEQSNWISDCLPALTANIRPDAVWVNRAES |
| | | FAQWQRGTQPGAQPPTPEEAQQILFSLCGESLVTLTLPEQPA |
| | | AAGQKQPDQETSPDPEEQTDRPPAAPSADDEMDPSNASRGI |
| | | FGDLFGENAEGKRSRSQGKDNFACAVRDFLCANPTPSADAI |
| | | TEFREKQKPREPNPPGPEKYPPEVSTSGAPTAVAKRYRKLL |
| | | VCAGLWPKSADEDGSSRNSAKTKFADPKEPQKTEIQINALD |
| | | LIDACNQAAPADDSGTSPKAGRVFAPAWASNIAEKVASAT |
| | | QMPANAKSLNEFKRLMFALAARRFSQTQSWTRRNEAERH |
| | | MAAARQDAAVARLREIDPDHKAQDWLRGYEQRRADQSGS |
| | | NGEFRITRRMIGEAEAVFKAWAGTNSAAERELKTVAVQTT |
| | | AEKFGDAALYSEIARNTAAEAVWRSGSAPEILDQWVKLRK |
| | | AQSDQQRTRVPRFCHPNAFRHPTWCEFGESSKPGVWYAWN |
| | | PKSKPRKPEVGGEGDGTRRLWVLLPDFNSGIGQAVPLRWRS |
| | | KRLSKDLGEALQPSDAPIPRADRVSIAAAGLNLEGANGVPA |
| | | RYRPSLPFSENTKGWNARLQANRTALLHLESKWDAEAATW |
| | | RDGGRSLLALKWFTTFSPELAMSEGPGRAIHPKLGWNSEPH |
| | | SDLNRAQKRGGNAKLILSRLPGLRVLSVDLGHRYAAACAV |
| | | WETLTTEQMNAACQAKNHTQPAESDMYVHLAHPTERVVK |
| | | SGRKKGQNLIQTTVYRRIAADTLPDGTPHPAPWGRLDRQFL |
| | | IKLQGEQRPTRAASKNEADLANALFHRLGLRSDADSENKSR |
| | | AVDKLMARTVRVATLGLKRHARRAKIAYALDPNTKAIPGM |
| | | GGSSAAFTPGDEPHIHLLTDALFDWQSLATDAKWDDAHAR |
| | | SLWNHHIATLPGGFHLENPTPRDESAHEPSRQRQRSGDDAL |
| | | RATLKPIAEKLSKADRQEVHAAWKKYWGDSDGQSAIVPKV |
| | | LQGQRGPEKTTPSASASGWHGKIRWITDWIMGKYLEGCTG |
| | | HAWKHDVGGLSVSRITTMKSLYQLHKAFAMRATPEKPRGA |
| | | PEKGESNLGAAQGILTAMESMRQQRVKQLASRIAEAALGA |
| | | GIERRSDNGRELQRPRERVDDPRFAACHAVVVEDLTNYRPD |
| | | EMQTRRENRQLMQWASSKVKKYLSEACQLHGLYLRGVPA |
| | | GYTSRQDSRTGAPGVRCGDIPVEELMAAPRWRRQILTAEKT |
| | | RRENNTGTARDRYILTLDEKYRLLTAEQRKKTPPARIPVKG |
| | | GDLFVSADPDSPAASGIQADLNAAANIGLKALIDPDWPGRW |
| | | WYIPCDATTHKPSPERTRGSAAVDCDVPLGPDSTGTPEDRD |
| | | AKPKKNQRNSKIAGRGQSAIINLWRDPTHLPIKENPSAWCES |
| | | KKYWNQVEHNVVKVIESKGQKLTQTAEAATGESASSPPIAP |
| | | TDVPW |

### Example 2: Exemplary Characterization of Additional Candidate Thermostable Cas Proteins

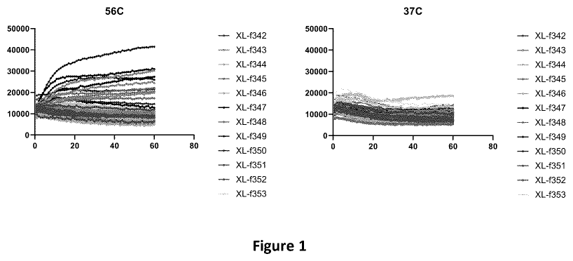

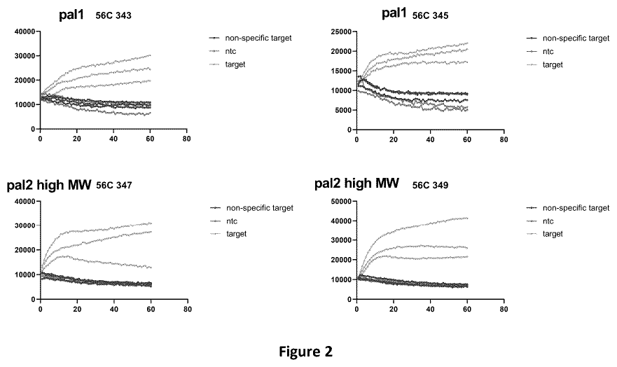

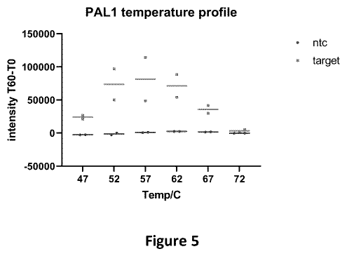

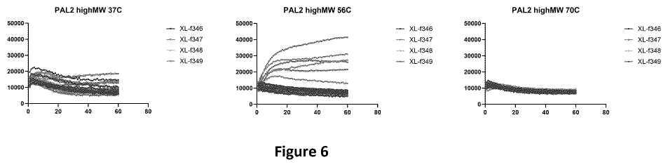

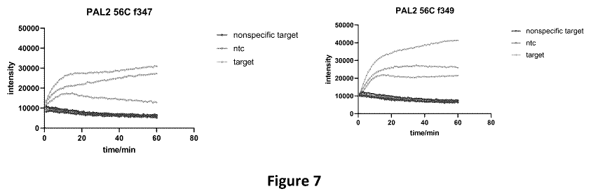

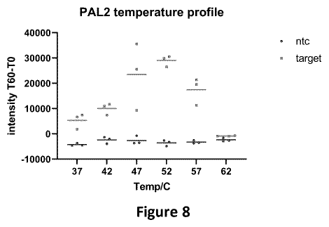

The present example demonstrates characterization of exemplary Thermostable Cas Proteins, Pal1 (SEQ ID NO. 1), Pal2 low MW, Pal2 high MW (SEQ ID NO. 2), and Pal3 (SEQ ID NO. 3). Each enzyme was tested with four guides (designated as 342-353) at both 37° C. and 56° C. in a Cas-only reaction with DnaseAlert as a reporter. Fluorescence signal was plotted vs. time for each reaction (FIG. 1). Pal1 showed low activity for two guides at 56° C. No activity was observed for Pal2 low MW or Pal3, while activity was observed for two guides at 56° C. for Pal2 high MW. Pal1 and Pal2 activity at 56° C. is shown in FIG. 2. Additional results of studies with these enzymes are shown in FIGS. 3-8. As can be seen, Pal1 showed activity for two guides at 56° C. and 70° C.; Pal1 showed maximum activity at 57° C. and significant activity to at least 67° C. Pal2 high MW showed activity for two guides at 56° C.; Pal2 high MW also showed maximum activity at 47-52° C. and significant activity up to at least 57° C. No significant activity was observed for Pal2 low MW, or any of Pal3-6 at 37° C., 56° C., or 70° C. Those skilled in the art thus understand that these enzymes are thermostable at least at about 56° C. and/or within a range of 56° C. and 70° C. These particular exemplified enzymes may also be described as thermoactive as their relevant activity(ies) are dramatically reduced and/or undetected at lower temperatures such as at 37° C. Without wishing to be bound by any particular theory, it is noted that enzymes of thermophilic organisms often may show reduced (or undetectable) activity at such temperatures.

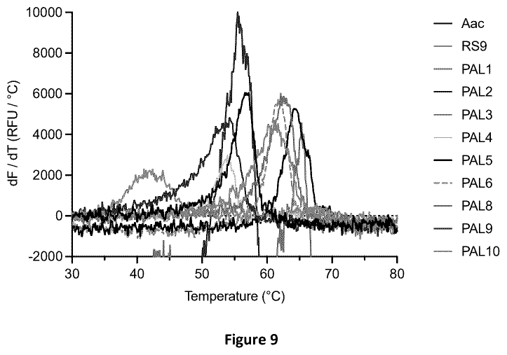

To further characterize exemplary Thermostable Cas Proteins, Pal1 (SEQ ID NO: 1), Pal2 (SEQ ID NO: 2), Pal3 (SEQ ID NO: 3), Pal4 (SEQ ID NO: 4), Pal5 (SEQ ID NO: 5), Pal6 (SEQ ID NO: 6), Pal8 (SEQ ID NO:8), Pal9 (SEQ ID NO: 9), and Pal10 (SEQ ID NO: 10), enzyme denaturation was evaluated using an exemplary protein melting method. Cas proteins were mixed with buffer and dye and a melt curve was run. As temperature increased, Cas proteins unfold. As exemplary Cas proteins unfold, hydrophobic regions are exposed, resulting in dye binding to the Cas proteins. Upon binding, the dye fluoresces. Change in fluorescence over change in temperature is plotted against the temperatures of the melt curve. Melting temperature of the Cas proteins was calculated and compared to that of an appropriate reference standard (e.g., Cas proteins with a known thermostability, e.g., Aac and/or RS9). Changes in melting temperature are correlated to changes in protein stability (e.g., thermostability) and activity (FIG. 9). FIG. 9 shows that Cas Proteins (PAL1-10) have a thermostable activity comparable to the reference Thermostable Proteins Aac and RS9, i.e. Cas Proteins PAL1-10 are thermostable.

### Example 3: Collateral Activity Signal of Thermostable Cas Proteins in Complex with Guide-RNA

The present example further demonstrates the collateral activity of Cas proteins described herein. The collateral activity of thermostable Cas Proteins (PAL5, PAL8, PAL9, and PAL10) complexed to different engineered guide-RNAs were tested in two different assays: an assay without target amplification (cas-only) and an assay with target amplification (RT-SLK). Guide-RNAs having different targets and lengths were engineered (SEQ ID NO's: 11-19). The engineered guide-RNAs are shown in Table 2.

TABLE 2
single guide-RNA (sgRNA) sequences for thermostable Cas12b
enzymes. sgRNA constant domain is in italic and spacer is in bold.

| SEQ ID | Sequence | | Enzyme/ |
|---|---|---|---|
| NO: | name | Sequence | PAL |
| 11 | crEF82 | GUUCCUACAGGGAACUCCAUUCCAAUGGGUG | 5 |
| | (SCoV2-N) | UGUCUUCCCCUUCAAUGGGUUUAGCACCAUU | |
| | | GGGUUGAUCAGAAUCUGAUCAACCGACAC | |
| | | CUCCUGCUAGAAUGGCUGGCAAUGGC | |
| 12 | crEF88 | GUUCCUACAGGGAACUCCAUUCCAAUGGGUG | 5 |
| | (SCoV2-O) | UGUCUUCCCCUUCAAUGGGUUUAGCACCAUU | |
| | | GGGUUGAUCAGAAUCUGAUCAACCGACA | |
| | | CCGUCUAUAAUCCGUUUAUGAUUGAU | |
| 13 | crJP97 | GCGCCUACAGGGCGCAUUCCAAAUCCACGACA | 8 |
| | (SCoV2-N- | UGUGUCUUCCCCUUCGUUGGGUUUAGCAUC | |
| | 1) | GUGGAGUCGAUCACACUCAGGUGUGCGGAGU | |
| | | GCAUGCACUUUGACAACUUGGCGCGCUGCGC | |
| | | GAGCGCGAAAACGCGCAGGCCAGCGCGCCGA | |
| | | UGAUCGACUGACAC | |
| | | CUCCUGCUAGAAUGGCUGGCAAUGGC | |
| 14 | crJP101 | CAAAUCCACGACAUGUGUCUUCCCCUUCGUU | 8 |
| | (SCoV2-N- | GGGUUUAGCAUCGUGGAGUCGAUCACACUCA | |
| | 5) | GGUGUGCGGAGUGCAUGGCGCGCUGCGCGA | |
| | | GCGCGAAAACGCGCAGGCCAGCGCGCCG | |
| | | CUCCUGCUAGAAUGGCUGGCAAUGGC | |
| 15 | crJP103 | GGCUAUAGGCCACAGGAAGCCACGGAAUGUG | 9 |
| | (SCoV2-N- | UCGUCCCCUUCAAUGGGCUUGGCACCGUGGC | |
| | 1) | GUCGAUCAGUUUUGUUCCGACUGAGGACAAA | |
| | | AGGUGCCGACGUCGGCACCAACCUGAUCGAC | |
| | | GGACAC | |
| | | CUCCUGCUAGAAUGGCUGGCAAUGGC | |
| 16 | crJP104 | GGCUAUAGGCCACAGGAAGCCACGGAAUGUG | 9 |
| | (SCoV2-N- | UCGUCCCCUUCAAUGGGCUUGGCACCGUGGC | |
| | 2) | GUCGAUCAGUUUUGUUCCGACUGAGGAAAAC | |
| | | CGACGUCGGCACCAACCUGAUCGACGGACAC | |
| | | CUCCUGCUAGAAUGGCUGGCAAUGGC | |
| 17 | crJP105 | GGCUAUAGGCCACAGGAAGCCACGGAAUGUG | 9 |
| | (SCoV2-N- | UCGUCCCCUUCAAUGGGCUUGGCACCGUGGC | |
| | 3) | GUCGAUCAGUUUUGUUCACCAACCUGAUCGA | |
| | | CGGACAC | |
| | | CUCCUGCUAGAAUGGCUGGCAAUGGC | |
| 18 | crJP107 | CCUCCUAAAGGGAGGCAGAACUCCGAGUAUG | 10 |
| | (SCoV2-N- | UGUCUUCCCCGUCAUUGGGCUUGGCACUCG | |
| | 1) | GGGUCAAUCUAAUUCACUUUGGAAAACCGAAG | |
| | | UGAAUCAGAUCGACCGACAC | |
| | | CUCCUGCUAGAAUGGCUGGCAAUGGC | |
| 19 | crJP109 | CCUCCUAAAGGGAGGCAGAACUCCGAGUAUG | 10 |
| | (SCoV2-N- | UGUCUUCCCCGUCAUUGGGCUUGGCACUCG | |
| | 3) | GAUUCACUUUGGAAAACCGAAGUGAAUGACAC | |
| | | CUCCUGCUAGAAUGGCUGGCAAUGGC | |

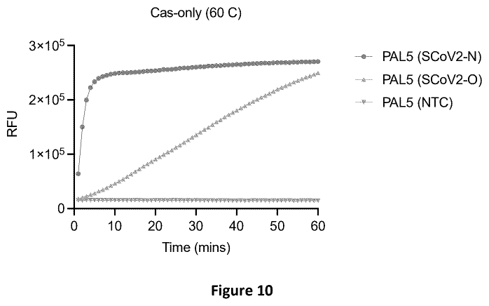

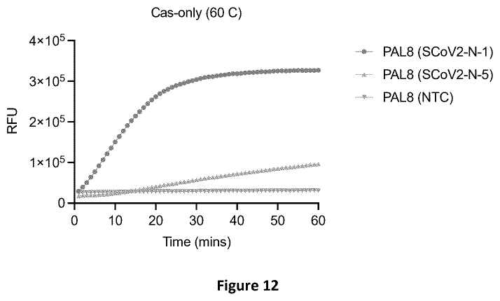

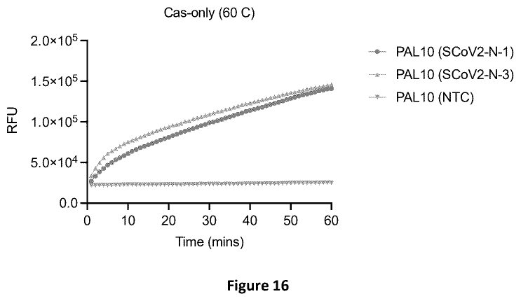

### Cas Only Reactions (FIGS. 10, 12, 14 and 16):

Cas only reactions comprise the relevant Cas enzyme complexed with an engineered guide-RNA that hybridized to its intended target, which is supplied as a pure gBlock DNA. In detail, for a Cas only reactions, amplification of target was performed separately from the Cas detection (Pal5 only). In some experiments a single stranded DNA target (oligo) was added directly to the Cas reaction at a concentration of 100 nM. For Pal5, LAMP-amplified target starting from 100 cp/uL (200 cp/reaction) of SARS-COV-2 genomic RNA was amplified using N gene or O gene specific LAMP primers in a 20 uL reaction (1× Warmstart RT-LAMP mix (NEB), 1× primer mix). The reaction as incubated at 60 C for 40 min. For all Cas only reactions, 5 μL of either ssDNA target or LAMP amplified product was added to 250 nM Pal-enzyme, 250 nM of respective guide, 8 mM MgCl2, and 250 nM DNAse Alert. Activation of the Cas enzyme was monitored at 60 C in a QS5 PCR machine and fluorescence was measured every minute.

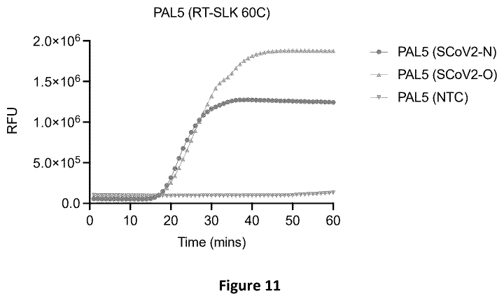

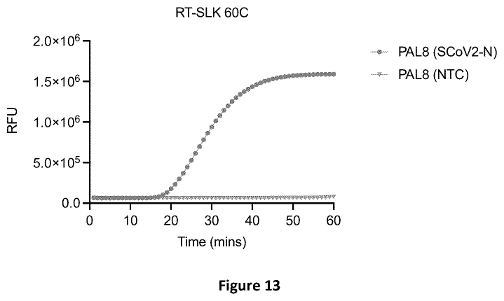

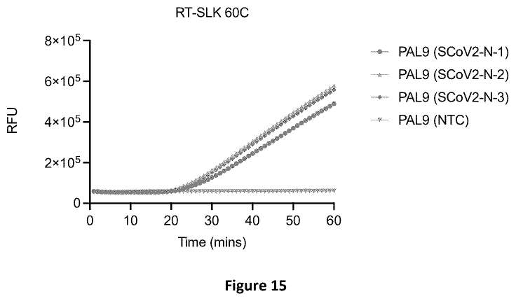

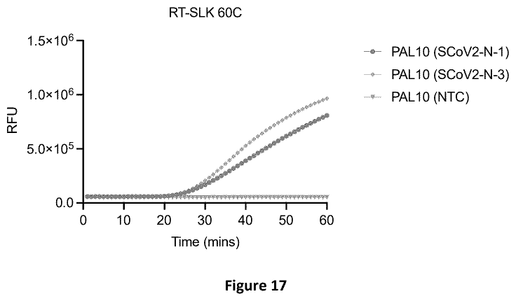

### Realtime SHERLOCK (RT-SLK) Reactions (FIGS. 11, 13, 15 and 17):

For RT-SLK reactions the target nucleic-acid is first exponentially amplified using LAMP, while the amplified material is simultaneously detected using a Cas enzyme in complex with its guide RNA. In detail, for RT-SLK reactions, LAMP-based amplification was coupled with Cas read-out in a single tube. Final concentrations for a 20 uL RT-SLK reaction are the following, 1× Warmstart RT-LAMP mix (NEB) was combined with 1× of the LAMP primers (primer set N or primer set O), 0.01 U/uL of TIPP (thermostable inorganic phosphatase (NEB)), 125 nM C7-FAM reporter or DNAse Alert (250 nM), 250 nM of Cas enzyme and 250 nM of respective guide RNA. As starting target material 100 cp/uL (200 cp/reaction) of SARS-COV-2 genomic RNA was used and the reaction was placed into a QS5 PCR machine, incubated at 60 C and the fluorescence was monitored and measured every minute.

FIGS. 10-17 show that the tested thermostable Cas Proteins (PAL5, PAL8, PAL9 and PAL10) are functional in a “one-pot” detection assay, which enables simple molecular diagnostics. Furthermore, FIGS. 11, 13, 15 and 17 show that thermostable Cas Proteins PAL5, PAL8, PAL9 and PAL10 are compatible with the chemistry enabling amplification (RT-SLK reactions), such as LAMP. The ability to combine amplification and detection in one-pot reduces the complexity of the overall diagnostic assay or therapeutic assay.

FIGS. 10 and 11 show that guide-RNA enables detection of multiple targets, here SCoV2-N or SCV2-O, by simply swapping out the variable domain of the guide (marked in bold in Table 2). Thus, PAL5 complexed to either guide-RNA crEF82 or guide-RNA crEF88 was able to detect its target. The two guide-RNAs had different performance levels but both showed unambiguous signal above background.

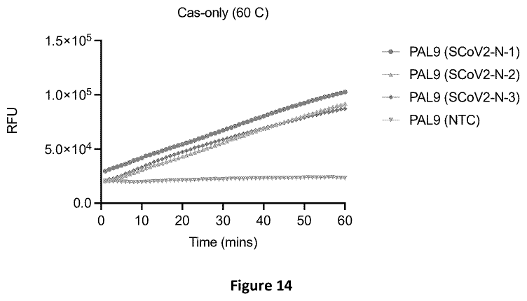

FIGS. 14-17 show that guide-RNA of various lengths complexed to PAL9 or PAL10 all retained full function (all showed unambiguous signal above background). This demonstrates that smaller guide-RNAs (e.g., crJP105, or crJP109) compared to longer guide RNAs (e.g., crJP103, crJP104, or crJP107) do not compromise functional activity in diagnostic or therapeutic assays.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A detection method comprising steps of:
contacting a CRISPR-Cas complex comprising:
a Cas protein with collateral cleavage activity that is thermostable at temperatures above at least 60-65° C.; and
a guide RNA selected or engineered to be complementary to a target nucleic acid sequence; with a sample potentially comprising a target nucleic acid sequence.

2. The method of claim 1, wherein the step of contacting comprises contacting the CRISPR-Cas complex and sample with a reporter susceptible to cleavage by the Cas protein collateral activity.

3. The method of claim 1, wherein the step of contacting comprises incubating for a period of time above the temperature.

4. The method of claim 1, further comprising a step of amplifying nucleic acid present in the sample.

5. The method of claim 4, wherein the step of amplifying utilizes a thermostable nucleic acid polymerase.

6. The method of claim 4, wherein the steps of amplifying and contacting are performed in a single vessel.

7. The method of claim 1, wherein the Cas protein is a Cas12 protein.

8. The method of claim 7, wherein the Cas protein has an amino acid sequence that is at least 80% identical to that of SEQ ID NO: 5, SEQ ID NO: 6, SEQ ID NOs: 8-10, SEQ ID NO: 7, or SEQ ID NOs: 1-4.

9-10. (canceled)

11. In a method of performing a detection assay utilizing a Cas protein with collateral cleavage activity, the improvement that comprises utilizing a Cas protein with thermostable collateral cleavage activity.

12. The improvement of claim 11, wherein the Cas protein is a Cas12 protein.

13-15. (canceled)

16. The improvement of claim 11, wherein the thermostable collateral cleavage activity is thermostable above a temperature of about 60° C.

17. The improvement of claim 11, wherein the thermostable collateral cleavage activity is thermostable above a temperature of about 65° C.

18. (canceled)

19. A non-naturally occurring or engineered composition comprising:
(a) a Cas protein with collateral cleavage activity that is thermostable at temperatures above at least 60-65° C.; and
(b) at least one guide capable of forming a complex with the Thermostable Cas Protein and directing the complex to bind to a target nucleic acid sequence.

20-21. (canceled)

22. The composition of claim 19, wherein the at least one guide comprises two guide sequences capable of hybridizing to two different target nucleic acid sequences or different regions of a target nucleic acid sequence.

23. The composition of claim 19, wherein the at least one guide comprises a plurality of guide sequences capable of hybridizing to a plurality of different target nucleic acid sequences or a plurality of different regions of a target nucleic acid sequence.

24. The composition of claim 19, wherein the guide sequence is capable of hybridizing to one or more target nucleic acid sequences in a prokaryotic cell or a eukaryotic cell.

25-52. (canceled)

53. A method of cleaving at least one target nucleic acid in a cell comprising contacting a cell with a Cas protein with collateral cleavage activity that is thermostable at temperatures above at least 60-65° C. and at least one guide capable of hybridizing to the at least one target nucleic acid, wherein the Cas protein is capable of forming a complex with the at least one guide and causing a break in the at least one target nucleic acid.

54-80. (canceled)


SEQUENCE LISTING (19 sequences)

SEQ ID NO: 1 - protein, 1335 aa, synthetic construct
MKSLAQFQNLYALQKTLRFELKPEGHTRETFNRWLEEIEKEQASENENIVYQDLLRAKKYEKIKIILDEYHKDFIEQALAYANLTELEKYEELYRKSNRTSEEEEEFENTKESLRKQIANIFIKNPNKTVQERWKFLFSKKLIQNELIVWVKGNYELLSEKLKNEFPDESSIISTIEDFKYFTTYFRNYHENRKNLYSNEDKFSTIAHRLIHENLPKFIDNIAIYQKAKAVLNINEVEKELGLPEDTLDKIFSLDFFSKALTQKGIDQYNYFLGGKTENEVKKIKGLNEFINLYNQQQQDKNQRLPFLKVLYKLPLFERTSTSFRFEPIENDRDLIERIGKFYYNDLKQYRDDSQGDTTDILSGINTLLRHVHDYRDGLYVNGGITLTQISQKIFGSWSYINNALAYFYDTYIDASGVDHQGERKPKKQKQIQEKTKWLKQKQFPVILVEKALSEYKSIETNEDLKTRISDTTLCDFFKRCGNDDNGQDLFDRIEARLREKNEEGYSLEDLLKKEFTTERKLMQDKTKTLLIKNFLDVIQGDKDDITAGLLHFVKCLIPRTEISEKNELFYSGMEKYLNILSEVTPLYNKARNYLTQKPYSIEKVKLNFENSTLLDGWDENEESDNSCVLLRKRGYYYLGIMNKKHNMIFDRKIYPKATEGEAYYEKMIYKLLPGAYKMLPKVFFSEKNIDYFKPSEEILRIRNTASYSKNGQPQEGYQKASFSIEDCRKYIDFFKKCIANHWDWQKFNFNFSPTEYYQSIDEFYREIERQGYKIDFVKIPESYINQLIKENKLYLFKIYNKDFSEKKKSKGKDNLHTLYWKMLFDEKNLKDVVLKLNGEAEVFFRQKSILYNEEIWNKGHHYSELKDRFSYPIISNKRYAEDKFFLHVPITLNFKADGINNVNNMVNEFIKDNRDIHIIGIDRGERHLLYVSVINQKGDIVEQCSLNEIVTEYNGKIFKKNYHEELDNLEKERDRARKDWQTIANIKELKEGYLSHVIHKISKLILKYNAIVVMEDLNSGFKRGRQKVEKQVYQNFEKQLIEKLNYLVLKESNVDEPGGVLRAYQLANKFETFKKLGKQSGIIFYVPAAYTSAIDPVTGYIQYLYPLKQADSVEKARKFYSQFKRISYNPHKQWFEFSFDYNDFNIIYHGKSSWTICTTNTERYMWNRLLNNGHGGEELVYVTNELELLFGEYNIIYGDGKDIKQQITDVQDIDVDRTAKQFYKRINELLNLTLKLRHNNGKKGADEEDYILSPVEPYFDSRFESRKPSMQQTLPINADANGAFNIARKGLLLLERLNQLGVEEFEKTKKSNNKKTQWLPHELWVEYAQNHTRK

SEQ ID NO: 2 - protein, 1273 aa, synthetic construct
MQDKTGWSSFTNKYSLSKTLRFELKPVGNTQKMLEDDGVFQKDRERQENYKKVKPFMDKLHREFIKEALNNLKLEGLTEYFEIFKKFRKDKNNKELKNAEKKLRQIIGRCYTETAQIWVEKYKEFGFKKKNIGFLFEEGVFELMKLKYGNDEASQIEKNGEVLSIFDGWKGFLGYFKKFFETRNNFYKDDGTSTAVSTRIINENLKIYLDNLIKYNKIKDKVDFKEADILQENKLNLSDFFNVESYAKYSLQKGIDYYNEILGGKTLKNGTKLKGLNEVINEYKQKNKSGELSKFKMLKKQILGEGEDRTLFEEIENEDELKDVLKDFFYNADPKITLFKTLLEDFFSNTEKYKDELDKIYFNTVAINGILHRWVDDSGVFQKYLFEVLKSNKLVKSNHYDKKEDSYKFPDFISFEHIKVALENCERDGLKDKFWKEKYYTKECLTENGLANLWQEFLEIYKCEFKKLYDYKTDDNDCYLQYRDNYKKYILDANFNPKEKSAKDIIKDYLDSVLSIYQLAKYFALEKKKVWTTDYETGDFYYEYIKFYEDTYEQIIKPYNLVRNYLTRKPINTAKKWKLNFDNAYLASGWDKDKEVSNLTVILRRDEQYYLAIMKKGKNKIFEKKFSCGEFEKMEYKQIAEASSDIHNLVLMNDGSCRRCIKMHDKRKYWPLDISIIKEKKSYAKENFVRRDFERFVNYMKKCSLLYWKEYDLKFSDTSTYKNINDFTNEIASQGYKLSFSAIPESYINEKNNNGELYLFQIYNKDFGIKTEGNKNLHTMYWESIFSEENRFRNFIVKLNGKAEIFYRPKSEQVEKEQRNFTREIIKNRRYTENKIYFHCPITLNRISRENVKKFNNGINNYIATNPNINILGVDRGEKHLVYYAIVDQDGKLIDAEDATGSFNTIGSTDYHRLLEEKAKDREKERKDWDLIRGIKDLKKGYISLVVRKIADLAIKYNAIIIFEDLNTRFKQIRGGMEKSVYQQLEKALINKLSFLVNKGEKDPEQAGHLLKAYQLAAPFQTFDKMGRQTGIIFYTQASYTSKIDPITGWRPNLYLKYRNIDDSKESIKKFKSILFNKEKNRFEFTYDLKDFVDFEEDKIPEKTEWTLCSSVERHKWNRHMNNNKGGYEVYKDLTENFYKLFDENNISMNKDIVDQVESISNGNFFRQFIYLFNLVCQIRNTDEKAEDVDKRDFILSPVEPFFDSRRAKDFKAYGDNLPKNGDENGAYNIARKGVLIIKKIKEYYNQNGSCDKLGWGDLSISHKEWDDFATNN

SEQ ID NO: 3 - protein, 1301 aa, synthetic construct
MDSYEQFTKLYPIQKTIRFELKPQGRTKEHFDNSNFLEKDRERDDNYKILKEVIDDYHREFIDECLSNIQLNWDDLKKFSEEYRRSKEKKNNRDSESEQKRMSTTSETRAINKKNLEAEQKRMRGEIVSAFKKDDRFKHLFSEKLFSILLKNQIYEKGTLEEIEAFDCFNKFSGYFKSFHENRKNMYSDEDKETAISYRIINENFPKLLDNFEKYQYVCREYPEQIREAESTLAEAGCYIKMDEIFSIDNFNNVMMQGGKESGISRYNLAIGGIVQGTGEKPKGLNEFLNLAYQNEPNGRKKIRMEPLYKQILSKEESFSYRLEAFTDDSQLLSAIRSFFDIVEKDKNGNIFDRAVNLMSSFSNYDTSKIYIRKAYLNQVSKEIFGYRGKSDSKPAKTADESLNKSGGWEKLGQMLRDYKADSIGDRNLEKTCKKVDKWLDSDEFTLSDILGAISLAGSNETFEAYVSEICVARRNIDKEKEKEKNINVEKISGDTESIQIIKALLDSVQEFFHLLSPFQLHPNTPHDWTFYAEFNDIYDKLSAITPLYNQARNHLTKKNLDTSKIKLNFNNPTLANGWDVNKEYENTAVILIRDGKYYLGIMNPKNKRKIKFDEGSGAGPFYQKMVYKLLPGPYRMLPKVFFAKKNIDYYNPSQEIREGYKAGKHKKGKEFDKGFCHKLIDFFKESIQKNENWKVFDFKFSPTESYDDISEFYQEVEKQGYRMYFVNIPSDTIDRYVEGGDMFLFQIYNKDFAKGAKGNKDMHTLYWNAVFSEENLQKGVMKLSGEAELFYRKKSDIKDPPHREGEILVNRTYIDRTHVSGVMGEQNTVKESRIPVPDEIHKNLFDYYNHGRELTKEEKEYCDKVGSFKAYYGIVKDRRYLENKMYFHVPLTLNFKAIGEKRINKMAIEKFLTDENACIIGIDRGERNLLYYSIIDRNGKIIDQKSLNVIDGFDYHEKLSQRQTEREVARQSWNSIGKIKDLKEGYLAKAVHEISKMAIKYNAIVVLEDLHFGFKKGRLKVEKQIYQKFEEMLINKLNYLVFKDVSDSSDAGGVLNAYQLTAPLESFSKLGKQSGILFYVPAAFTSVIDPTTGFVDLFNSSSITSTQKKKEFLQRFESIVYSARDGGIFAFTFDYRNFSKIATDHRNMWTVYTHGERIRYVRDEKCYKTTDPTKRIKEALSGIEYDDGSDIRDKITQSGDNNLINTVYHSFMDTIKMRNKDGRIDYIISPVKNRNGEFFRSDYKHRDFPVDADANGAYHIALKGELLMRMIGKTYDSNSDKMPKLEHKDWFEFMQTRGDQ

SEQ ID NO: 4 - protein, 1188 aa, synthetic construct
MCVSRLPWFNITLTGKLNRQRLNQMCVSRLPWFCTPKGQLAATPKTVVAQQENAMLAIIRDVHEAAPADLKTVAQRLEPGYFVTQFPKQQMTGDEARAEAERLFAACQKKFKELAEYEDGYRQCLDALGPNLSLPRLGRKPKGAYPYAVVFKLMPTNATWECFKRVTASLYKRAQKGVVSPVSADSIADVRTNDEPLFEYFTNLALVRPPGNKDRAVWFEFDLAAFIEAIKSPHQFFQDTIKREQAVAQIKAKLDAMDGQGRAASGEEDALPGFEGDDRITLLRELVTDTLGYLAEADASTSPGGKIEYSIQERTVRGFAEVKRRWRDLVEKGKATEDALLKVLAEEQTEHRDDFGSATLYRELAKPKFQPIWRDPGTQPWHADDPLRAWLEYRELGRELEDKQRPIRFTPVHPVHSPRFFIFPKKKGGGRFGTVHEPGQLRVMAGIVAQTQHGWEPVPVRITYAAPRLRRDQLRDDVETDLESRPWLQPMMQALGLPEPDTADFSNCRVTLQPSAPDDIQLTFPVDVSADKLTTAIGKAARWAKQFNLFPDGDNFYNASLRWPHEKKPSKPPVPWHEALDNFSVLAADLGQRCAGAFARLEVRANDDFAGKPSRFIGETPGKKWRAALVAAGMLRLPGEEQTVWRPGATGPNFHTELSGSRGRMARPHEADDTADLLRAFDCPEESLMPADWRTSLSFPEQNDKLLVAARRYQSRLARLHRWCWFLTDEKKRQTALDEIREAEDMPAADDPQLTDKLRALLLQKQAALPGLLVRLANRILPLRGRSWQWETHPDKADCHLLTQTGPALPDVWIRGQRGLSMQRIEQIEELRRRFQSLNQMQRREIGGKPPIRRDDSIPDCCPDLLDKLDQIKEQRANQAAHMILAEALGLRLAPPPADKRQLRASRDVHGQYVKSREPVDFIVIEDLSRYRSSQGRAPRENSRLMKWCHRAVRDKLRELCEPFGIPVVETPAAYSSRFCSRSGVAGFRAVEVGPGFDREFPWMMLKDREDEGEPVRQLILQVATLNQGRDGKPPRTLLAPLAGGPIFVPIVDKLNGADIQPALAQADINAAINLGLRAIADPRLWSIHPRCRTQRQGDQMLTREKRKFGETGQPLAVHRADGVKPDDTRNPNFFADISGSLPAWESATLDGQHLLSGRCLRSEIKKRQWQRCAEINDRRMNRWMKGE

SEQ ID NO: 5 - protein, 1518 aa, synthetic construct
MTELQTQRAYTLRLKGIDEKDQSWRDALWKTHEAVNKGAKVFGDWLLTLRGGLDHTLADAEIPGEKGKPDRAPTQEERKHRRILLALSWLSVESERGAPEEFIVATGKEPAATRNDKVIAALKDILRGRNLTEEKISEWTEVCTPSLSAAIREDAVWVNRSRAFDEAVKRIGSSLTREEVWDMLECFFGSRNAYLAPVKISEDESSDGEQEEKAKDLVQKAGQWLSSRFGTGEGADFAKMAAVYAKIAAWAGNAQAGTTGNEVINNLATALREFTPKSNDLKGVLDLISGPGYKSATRNLLKQIANTKTVTREDISKLQETAGEDSEECATKTGSKGKRAYADAILKDVESVCGFTYRIDKDGQPVSVADYSKYDEDYKWGSSRHKEFAVMLDHAARRVSLAHTWIKRAEAERRKFEEDSKKIMQVPQAAKDWLDAYCAQRSEASGALEPYRIRKRAIQGWKEIIASWNKPDCKTAEDRIAAARQLQDDPEIEKFGDIQLFEALAEDDAQCVWKKEDGTLDPEILINYTLASEAMFKKQHFKVPSYRHPDAFLYPVFCDFGNSRWELDFSIREAATKLKEIEAKIEKQRQEVHKVQQALEKCENDEKRPKMEERLKEAQKKLQESQNYGEYLHSNNKITMVLFDGTFVKKHIFAWQSKRLTKDLALYQEPSADPKNVVSRADRLGRAVASVGINDAVKVAGLFEQENWNGRLQAPRQQLEAIAQYVEKHGWDNKAEKMRASIKWFITFSAKLQSKGPWNEFARKHGLKEDPHYWPHAEKNENRTAHSRLILSRLPGLRVLSVDLGHRYAAACAVWEALGSEAFKKDIEGKRIIRGDTDENALYCHTEHEANGKKHITIYRRIGADTLPDGAHHPAPWARLDRQFLIKLQGEDEQAREASNEEIWKVHQLENTLGRRTPLIDRLIAGGWGYTEKQKARLEVLTNLGWCPTNKTDNQEEGDEEETAILSKPSLLVDDLMFSAVRTLRLALKRHGDRARIAHYLITDEKTKPGGVKEKLDKNGRVELLLDALGLWHDLFSSPGWHDEKAKQLWNAYIAGLLPEGELQQAKSVTTSAALGGQQKKEKKEKLRAVAEALYLNSDLCHSLNEVWRKRWEEDDKQWRIYIRWFKDWIMPRGANAKSPAIRHVGGLSLTRLATLTEFRRKVQVGFFTRLHPDGTKTETREDFGQKTLDTLEHLREQRVKQLASRIVEAALGIGSEDKRHWDGKKRPRQRIADPRFVPCHAVVIENLTHYRPEETRTRRENRQIMEWASSKVKKYLSEICQLHGLHLREVSAAYTSHQDSRTGAPGIRCQDVSLIEFMKSPFWRKQVAQAEKKQKEGKGDAVERYLCELNQKWKGASEEEWRKAGFVRIPLRGGEIFVSAAGHDSPAAKGIHADLNAAANIGLRALLDPDWSGKWWYVPCNSSTMCPARDKVTGSAAVNPGQPLQVSAQLESDDAAKDTKKRKKKGDGKSKEIINLWRDISSYPLEDTRGGTWSNKTVYWNRVQSNVVHILQNQMKG

SEQ ID NO: 6 - protein, 1374 aa, synthetic construct
MPETTQRAYTLRLQGHDPKDASWREALWKTHEAVNRGAKAFGDWLLTLRGGLDHSLADEGAPGQTPTEEQRKQRRILLALSWLSVESENGAPQEYIVPHDRDNESGARQNWKTREALREILKNRGCRDDEIESWCHDCEPSLTSAIRKDAVWVNRSKAFDNAVQSIPNFSREEIWDLLGCFFVSSQAYLAPLESPKDDKPDASKKDSSKDLIQSAGQWLSRRFGRGKGLNFARLAETYEAIARWASVANPGDTNDLIADLAKTLNAETPELDGILKVVSGPGHKSKTRNLLRSLSAVNHITKDTLQRLKDTANEDAKKAKLKKGEKGHRAYAYKVLEAVEDACGFTYLQEGDRAKHCEFAVMLDHAARRVSSLHTWIKRAEAERRRFEIDTKKKDQLPPSVKEWLDTYCQKRSKETGAVEPYRIRRGAIEGWKEIVEAWSKAGTTTAEDRKHEARRLPDNPHIDKSGDIKLFEDLALEDALPVWHANGDPNNPPDPQLLIDYVEGSEAEFKKRAFKVPTYCHPDPLVHPVFCDYGCSRWNVSFAIQPVKKQKLSSEEKLPAKGLLLDLLHGTAIRPVALRWQSKRFARDLALNTTDSSDKPNEVTRADRFGCALAKCPSSQKIRIRGLFEEKYWNGRLQAPRPELTALAKRVAKYGWDKKARKLRNSLNWFITFSANLRPSGPWEEYTKYAEKAFSSNASAKPSVSRGGFWVVHASPNKRGKMAQLRLCRLPELRVLSVDLGHRYAAACAVWETLSKSAFEQEIHERKILRGGTGPNDLFCHTQHDTNGQSKVTIYRRIGADTLPNGTPHPAPWARLDRQFLIKLPGEEREARKASPTELANVEKLEKELGLKTSENRVKRIDDLMSDTLRTVRQALRRHSLRARIAFNLATLRDQSDGDEESQSKQKRDTRWNNTVKIWHSLLESNEWTDDWAKALWDELGPLSDPQKADDAEWLKLAAEKFYTRWQEDEQTWRERLRWLRRWILPRGSQAASQKGSIRHVGGLSLTRLATIKTLYQVLKAYHMRLKPDNSRKNIPAEGDEALQNFGQKILDDLEHMREQRVKQLASRIVEAALGLGRMKQVTIGKDPKRPREPVDQSCHAVVIENLTHYRPEKRQTRRENRQLMDWSAAKVKKYLKECCQLHGLHLVEVSASYTSRQDSRTGAPGIRCQEVPLTDFLKKNFWREQVKQAKQRLSEGKANARDRYLCQLNERWGNAPAPVTQTAIRLRIPLNGGELFVSADQNSPASKGIQADLNAAANIGLRAITDPDWPGAWWYVPCEANTFKPVKDKVAGSAAIDSNVSLKKDSPNSEKPASDRKSRTSKSMINLWCDTSSKSLSEKDQWQESAPYWEDVAARTINILQASLACSTTNSQ

SEQ ID NO: 7 - protein, 1335 aa, synthetic construct; identical to SEQ ID NO: 1

SEQ ID NO: 8 - protein, 1520 aa, synthetic construct
MAYQNGKEQPTVTNQRAYTLRLSGTNDQDSIWRNRLWHTHEAVNKGAKTFGDWLLTMRGGLCHTLAEADVPGKGNKPARHPTPQEIRSRRVVLALSWLSVESQHGAPERHLVSHDLDIATGERKNWKTVEALREILHGRCLCKELIDEWANDCRDSLSATIREDAVWVNRSKAFDLAAKKIGASLTREELWDFLQPFFANKHGYLQMDTVAGVTNGDSETDAEEAKEDSSEEKAKDLSQKAGQWLSSRFGTGTGADFSRFSKVYEVLAARCGSVAVGVSGVEAIRILAGTLADFSPGSNDIEGMLGLMSGPGYKSATRNILQKINTLQTVSQQDLDRLREASEKDALQSKQKVGGKGSRPYANAILQDVEAACGICYAGTGESPARHWQYAVILDHAARRVSMAHSWIKRAEEQRSKFEIEKDKLDHVPKDALAWLDAFCARRSSESGASDAYRIRRSAVDGWKQVVAAWAALPPKPENQGSELLSDAESARIQAARELQDTVEKFGDIQLFEALSLTGAKCVWQPDGRPDAQPLLDYVAGTDAISKKQRFKVPAYRHPDALLHPVFCDFGNSRWNINYAIHRAPEKLTPAQQLLEKKKAEIDKAELTLAKAGDAAKQANISEKINGLRAAFIQQQEKVAWLNSRHAMTMSLWDGTHIEDTPLIWQSKRFGSDIGQPVEAQPLPVSRADRFGRAVALAQDNVPVIPSGLFDLSDWNGRLQAPRRQLEAIAAIRDSAKLSVNEKQQLVAKRIQSIRWLLTFSAKLQSHGPFIAYAAQHGFDWRYGAHGPENKSRQGLAKLILCRLPGLRILSVDLGHRYAAACAVWETLNAGQIQKACLDAGKEAPGPCTLYLHLKQIANGKEKKTIFRRIAADTLPDGSPHPAPWARLDRQFLIKLQGEDRDARLATSEEIAAVEQMENELGVVRQLKRKGRELLVDELMSDALRTLRLGLRRHGVRARIAFNLTANKRIRPGGKEEVLDQEGRVLLLTETLLAWYELYTAERWTDEPARELWNRHIQPLLGATILQNTVNQEDTPSAAKRRKLREETSGKLKHVAEEIAKNDSLCRQLHVLWSAQWQTEDVIWRTRLRMMRRWLLPRGVKRNAQLRISIRDVGGLSLTRIASFKSLYQVQKAYQMRPHPEDPRLNIPERGDSRLENFGQRVLDAMERMRENRVKQLASRIAEAALGIGGETGISSKDGSQKKRPTERSSDPRFAPCHAVVIEDLTHYRPDETQTRRENRQLMSWSSSKVKKYLGEACELNGLYLREVSPAYTSRQDSRTGAPGLRCNDVTVVEFNNSPFWRKQVGAAEKNQKEGNKGDARERYLLSIEEGIRGAANDRDIFRIPVKGGEIFVSACITDGGNNAKKNAPPGLQADLNAAANIGLRAIFDPDWEGRWWYIPCDAATLCPDAKKFIGCKAVDPTKPLRVVAEEGAISASGIGSKKSGRKKNAATDGTRIVNLWRDPSGAPIHRDVLRSPEWQDYAGYWNEVQHRVIRNLKTCYEQTSQQEDPFVSQDADKPF

SEQ ID NO: 9 - protein, 1180 aa, synthetic construct
MKRLAETALADKVKCETNSRPKGERAYANSILHDVESACGFTYRVDKGEQPVPVSDYSHYANDYRWGPANHSEFAVMLDHAARRVSLAHTWIKRAEAERRQFEENAKKIDKVPKVAREWLDSLCAERSIVLGALEPYRIRRRAVDGWKHVVAAWSKSDCKTAQDRITAARLLQEDPEIDKFGDIQLFEALAEDHAVCVWQRDGEAGKTSDPQLLIDYALAAEAEFKKRHFKVPAYRHPEAFWHPVFCDFGQSRWKICFDVHKNRQSRRQRACANRISRKICFDVHKKRQTLRLSLEVWTGSKMLDMPLCWQCKRLARDLALGQDHKKDRSCQVTRADRLGRAVSNVARNQEVQILGLFEQEYWNGRLQAPRPQLEALGRYIEKHGWDAKAQKSCRAIRWMISFSPRLQPAGPWGKFAEKLQLNPNPKYWPHAEDNKDRGSRSKLILCRLPGLRVLSVDLGHRYAAACAVWEAVDAEQVKEACQAAGHREPNENDLYLHLKKRTTKQKKGSQGVVEETTIYRRIGADTLPDCTPHPAPWARLDRQFLIRLQGEEDEARAASNEEVWAVHKLEAELGRTIPLIDRLLGAGWGQTEKQKARLKALRELGWTPANKCQAFNSTDETELRRPSLAVDELMLDAVGTLRLALKRHGDRARIARYLITDERTKPGGVKEKLDENGRIELLQDALIIWHGLFSSPRWRDDAAKQLWNEHIAKLVGEQNLVEVSEDASGSERRTKQKQNREKLREAAKALVDDVALRQALHDMWKRRWEEEDREWRRRLRWFKDWVLPRREQARKAYSRPAETGSSSHPKRRARYAAIRRVGGLSLTRLATLTEFRRKVQVGFFTRLKPDGTKAEAKEGFGQSTLDALEHLRAQRVKQLASRIVEAALGVGRIRRFPGVKNPKRPDTPVDKPCHAIVIENLTHYRPEETRTRRENRQLMTWSSSKIKKYLAEACQLYGLHLREVTAAYTSRQDSRTGAPGLRCQDVPVKEFMRSLFWRKEVAQAEKKLTAGKGSSYERLLCELNQRWKDNSPGDGKRAELLRLPHKGGEIFVSAAPDSPAARGLQADLNAAANIGLRALTDPDWPGKWWHVPCNAVTFRPVEDKVKGSAAVKLDQSLRQVAHPQSKDPGAKKSKEIVNLWCDISSLPLEHREWKLDWEPYPAYWNNVQCRVIRVLQGKV

SEQ ID NO: 10 - protein, 1546 aa, synthetic construct
MANAKVKTTTRSYTLSLNAPSDTTDRSPLWHRIFRTHYAICCGAREFGKLLLDLRGGLPTSLAQLGEGIAENDRRQTQRGTRRILALGWLSVEDLDHARNDPHRVQDTAPGSPLDQDLAEKILRKILITKGIKSEEEQSNWISDCLPALTANIRPDAVWVNRAESFAQWQRGTQPGAQPPTPEEAQQILFSLCGESLVTLTLPEQPAAAGQKQPDQETSPDPEEQTDRPPAAPSADDEMDPSNASRGIFGDLFGENAEGKRSRSQGKDNFACAVRDFLCANPTPSADAITEFREKQKPREPNPPGPEKYPPEVSTSGAPTAVAKRYRKLLVCAGLWPKSADEDGSSRNSAKTKFADPKEPQKTEIQINALDLIDACNQAAPADDSGTSPKAGRVFAPAWASNIAEKVASATQMPANAKSLNEFKRLMFALAARRFSQTQSWTRRNEAERHMAAARQDAAVARLREIDPDHKAQDWLRGYEQRRADQSGSNGEFRITRRMIGEAEAVFKAWAGTNSAAERELKTVAVQTTAEKFGDAALYSEIARNTAAEAVWRSGSAPEILDQWVKLRKAQSDQQRTRVPRFCHPNAFRHPTWCEFGESSKPGVWYAWNPKSKPRKPEVGGEGDGTRRLWVLLPDFNSGIGQAVPLRWRSKRLSKDLGEALQPSDAPIPRADRVSIAAAGLNLEGANGVPARYRPSLPFSENTKGWNARLQANRTALLHLESKWDAEAATWRDGGRSLLALKWFTTFSPELAMSEGPGRAIHPKLGWNSEPHSDLNRAQKRGGNAKLILSRLPGLRVLSVDLGHRYAAACAVWETLTTEQMNAACQAKNHTQPAESDMYVHLAHPTERVVKSGRKKGQNLIQTTVYRRIAADTLPDGTPHPAPWGRLDRQFLIKLQGEQRPTRAASKNEADLANALFHRLGLRSDADSENKSRAVDKLMARTVRVATLGLKRHARRAKIAYALDPNTKAIPGMGGSSAAFTPGDEPHIHLLTDALFDWQSLATDAKWDDAHARSLWNHHIATLPGGFHLENPTPRDESAHEPSRQRQRSGDDALRATLKPIAEKLSKADRQEVHAAWKKYWGDSDGQSAIVPKVLQGQRGPEKTTPSASASGWHGKIRWITDWIMGKYLEGCTGHAWKHDVGGLSVSRITTMKSLYQLHKAFAMRATPEKPRGAPEKGESNLGAAQGILTAMESMRQQRVKQLASRIAEAALGAGIERRSDNGRELQRPRERVDDPRFAACHAVVVEDLTNYRPDEMQTRRENRQLMQWASSKVKKYLSEACQLHGLYLRGVPAGYTSRQDSRTGAPGVRCGDIPVEELMAAPRWRRQILTAEKTRRENNTGTARDRYILTLDEKYRLLTAEQRKKTPPARIPVKGGDLFVSADPDSPAASGIQADLNAAANIGLKALIDPDWPGRWWYIPCDATTHKPSPERTRGSAAVDCDVPLGPDSTGTPEDRDAKPKKNQRNSKIAGRGQSAIINLWRDPTHLPIKENPSAWCESKKYWNQVEHNVVKVIESKGQKLTQTAEAATGESASSPPIAPTDVPW

SEQ ID NO: 11 - RNA, 117 nt, synthetic construct
  source 1..117: mol_type = other RNA
gttcctacagggaactccattccaatgggtgtgtcttccccttcaatgggtttagcaccattgggttgatcagaatctgatcaaccgacacctcctgctagaatggctggcaatggc

SEQ ID NO: 12 - RNA, 116 nt, synthetic construct
  source 1..116: mol_type = other RNA
gttcctacagggaactccattccaatgggtgtgtcttccccttcaatgggtttagcaccattgggttgatcagaatctgatcaaccgacaccgtctataatccgtttatgattgat

SEQ ID NO: 13 - RNA, 195 nt, synthetic construct
  source 1..195: mol_type = other RNA
gcgcctacagggcgcattccaaatccacgacatgtgtcttccccttcgttgggtttagcatcgtggagtcgatcacactcaggtgtgcggagtgcatgcactttgacaacttggcgcgctgcgcgagcgcgaaaacgcgcaggccagcgcgccgatgatcgactgacacctcctgctagaatggctggcaatggc

SEQ ID NO: 14 - RNA, 146 nt, synthetic construct
  source 1..146: mol_type = other RNA
caaatccacgacatgtgtcttccccttcgttgggtttagcatcgtggagtcgatcacactcaggtgtgcggagtgcatggcgcgctgcgcgagcgcgaaaacgcgcaggccagcgcgccgctcctgctagaatggctggcaatggc

SEQ ID NO: 15 - RNA, 156 nt, synthetic construct
  source 1..156: mol_type = other RNA
ggctataggccacaggaagccacggaatgtgtcgtccccttcaatgggcttggcaccgtggcgtcgatcagttttgttccgactgaggacaaaaggtgccgacgtcggcaccaacctgatcgacggacacctcctgctagaatggctggcaatggc

SEQ ID NO: 16 - RNA, 150 nt, synthetic construct
  source 1..150: mol_type = other RNA
ggctataggccacaggaagccacggaatgtgtcgtccccttcaatgggcttggcaccgtggcgtcgatcagttttgttccgactgaggaaaaccgacgtcggcaccaacctgatcgacggacacctcctgctagaatggctggcaatggc

SEQ ID NO: 17 - RNA, 126 nt, synthetic construct
  source 1..126: mol_type = other RNA
ggctataggccacaggaagccacggaatgtgtcgtccccttcaatgggcttggcaccgtggcgtcgatcagttttgttcaccaacctgatcgacggacacctcctgctagaatggctggcaatggc

SEQ ID NO: 18 - RNA, 139 nt, synthetic construct
  source 1..139: mol_type = other RNA
cctcctaaagggaggcagaactccgagtatgtgtcttccccgtcattgggcttggcactcggggtcaatctaattcactttggaaaaccgaagtgaatcagatcgaccgacacctcctgctagaatggctggcaatggc

SEQ ID NO: 19 - RNA, 119 nt, synthetic construct
  source 1..119: mol_type = other RNA
cctcctaaagggaggcagaactccgagtatgtgtcttccccgtcattgggcttggcactcggattcactttggaaaaccgaagtgaatgacacctcctgctagaatggctggcaatggc